(19) United States
(12) Patent Application Publication
(10) Pub. No.: US 2022/0183633 A1
(43) Pub. Date: Jun. 16, 2022

(54) DETECTION AND/OR PREDICTION OF STROKE USING IMPEDANCE MEASUREMENTS

(71) Applicant: Covidien LP, Mansfield, MA (US)

(72) Inventors: Patrick W. Kinzie, Glendale, AZ (US); Randal C. Schulhauser, Phoenix, AZ (US); Aaron Gilletti, Chandler, AZ (US); Scott J. Schuemann, Wildwood, MO (US); Daniel Hahn, Tustin, CA (US); John Wainwright, Foothill Ranch, CA (US)

(21) Appl. No.: 17/644,129

(22) Filed: Dec. 14, 2021

Related U.S. Application Data
(60) Provisional application No. 63/126,310, filed on Dec. 16, 2020.

(51) Int. Cl. A61B 5/00; A61B 5/369; A61B 5/318; A61B 5/11; A61B 5/029; A61B 5/0205

(52) U.S. Cl. CPC A61B 5/7275; A61B 5/369; A61B 5/746; A61B 5/318; A61B 5/1117; A61B 5/4803; A61B 2562/16; A61B 5/6822; A61B 5/6823; A61B 5/686; A61B 5/029; A61B 5/0205; A61B 5/6814

(57) ABSTRACT

A system comprises a memory, a plurality of electrodes, sensing circuitry, and processing circuitry. The sensing circuitry configured to determine one or more tissue impedance values via the electrodes, wherein the tissue impedance values vary as a function of ejection fraction of a heart of a patient. The processing circuitry configured to determine, at least based on the one or more tissue impedance values, a stroke metric indicative of a stroke status of the patient, and store the stroke metric in a memory.

This application claims the benefit of U.S. Provisional Application Ser. No. 63/126,310, filed Dec. 16, 2020, the entire content of which is incorporated herein by reference.

### TECHNICAL FIELD

This disclosure is directed to medical devices and, more particularly, to systems and methods for detecting and/or predicting stroke.

### BACKGROUND

Stroke is a serious medical condition that can cause permanent neurological damage, complications, and death. Stroke may be characterized as the rapidly developing loss of brain functions due to a disturbance in the blood vessels supplying blood to the brain. The loss of brain functions can be a result of ischemia (lack of blood supply) caused by thrombosis, embolism, or hemorrhage. The decrease in blood supply can lead to dysfunction of the brain tissue in that area.

Stroke is the number two cause of death worldwide and the number one cause of disability. Speed to treatment is the critical factor in stroke treatment as 1.9M neurons are lost per minute on average during a stroke. Stroke diagnosis and time between event and therapy delivery are the primary barriers to improving therapy effectiveness. Stroke has three primary etiologies: i) ischemic stroke (representing about 65% of all strokes), ii) hemorrhagic stroke (representing about 10% of all strokes), and iii) cryptogenic strokes (representing about 25% of all strokes, and including transient ischemic attack, or TIA). Strokes can be considered as having neurogenic and/or cardiogenic origins.

A variety of approaches exist for treating patients undergoing a stroke. For example, a clinician may administer anticoagulants, such as warfarin, or may undertake intravascular interventions such as thrombectomy procedures to treat ischemic stroke. As another example, a clinician may administer antihypertensive drugs, such as beta blockers (e.g., Labetalol) and ACE-inhibitors (e.g., Enalapril) or may undertake intravascular interventions such as coil embolization to treat hemorrhagic stroke. Lastly, if stroke symptoms have been resolved on their own with negative neurological work-up, a clinician may administer long-term cardiac monitoring (external or implantable) to determine potential cardiac origins of cryptogenic stroke.

### SUMMARY

In general, the disclosure is directed to devices, systems, and techniques for detecting and predicting stroke via one or more medical devices, e.g., implantable medical devices (IMDs) or external medical devices, which may be located on or near the head of a patient. For example, an IMD may include a plurality of electrodes carried by a housing of the device. The IMD may be implanted subcutaneously in a region of the thorax, on the back of the neck, or in a region of the cranium. From this location, the IMD may be able to record electrical signals from the electrodes carried on the housing. These electrical signals may contain components attributable to brain function and components contributable to cardiac function. The IMD may be able to measure impedance signals that vary based on cardiac performance and/or brain electrical activity via the electrodes. The IMD may process the electrical signals to determine stroke metrics indicative of the risk of stroke of the patient. Therefore, the IMD may be able to detect or predict stroke events for the patient from a single device. The IMD may transmit information representative of any detected or predicted stroke to an external device. In other examples, processing circuitry may detect or predict stroke events based on signals sensed by two or more implanted or external devices.

The techniques of this disclosure may provide one or more advantages. For example, it may be beneficial for a system to be able to detect and predict the risk of stroke using brain, cardiac, and motion signals sensed via a single sensor device. Such a device may be relatively unobtrusive and usable for extended periods during patient daily living when compared to other devices typically employed to detect stroke, e.g., devices used in a clinic, or devices prescribed to provide treatment for stroke. The sensor device is configured to sense both brain and cardiac features from its position, and additionally sense a motion signal to further enhance its ability to detect and predict the risk of stroke. In some examples, the sensor device may communicate with additional devices including additional sensors sensing additional signals (e.g., motion sensors, heart rate sensors, or electrocardiogram sensors from a phone, watch, or other wearable device), which may allow improving the sensitivity and specificity of algorithms used to detect and predict the risk of stroke for the patient.

In one example, a system includes a memory; a plurality of electrodes; sensing circuitry configured to: determine one or more tissue impedance values via the electrodes, wherein the one or more tissue impedance values vary as a function of ejection fraction of a heart of a patient; and processing circuitry configured to: determine, at least based on the one or more tissue impedance values, a stroke metric indicative of a stroke status of the patient; and store the stroke metric in the memory.

In another example, a method includes determining, via a plurality of electrodes, one or more tissue impedance values, wherein the tissue impedance values vary as a function of ejection fraction of a heart of a patient; determining, at least based on the one or more tissue impedance values, a stroke metric indicative of a stroke status of the patient; and storing the stroke metric in a memory.

In another example, a computer readable storage medium includes instructions that, when executed, cause processing circuitry to perform any of the methods described herein.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1A is a conceptual diagram of a system configured to detect and predict the risk of stroke in accordance with examples of the present disclosure.

FIG. 1B is a conceptual diagram of a system configured to detect and predict the risk stroke in accordance with examples of the present disclosure.

FIG. 1C is a diagram of the 10-20 map for electroencephalography (EEG) sensor measurements.

FIG. 2A depicts a top view of a sensor device in accordance with examples of the present disclosure.

FIG. 2B depicts a side view of the sensor device shown in FIG. 2A in accordance with examples of the present disclosure.

FIG. 2C depicts a top view of another example sensor device in accordance with examples of the present disclosure.

FIG. 2D depicts a side view of another example sensor device in accordance with examples of the present disclosure.

FIG. 2E depicts a side view of another example sensor device in accordance with examples of the present disclosure.

FIG. 2F depicts a side view of another example sensor device in accordance with examples of the present disclosure.

FIG. 2G depicts a top view of another example sensor device in accordance with examples of the present disclosure.

FIG. 2H depicts a top view of another example sensor device in accordance with examples of the present disclosure.

FIG. 21 depicts a top view of another sensor device with electrode extensions to increase a sensing vector size in accordance with examples of the present disclosure.

FIG. 3A-3C depicts other sensor devices in accordance with examples of the present disclosure.

FIG. 4 is a block diagram illustrating an example configuration of a sensor device.

FIG. 5 is a block diagram of an example configuration of an external device configured to communicate with the sensor device of FIG. 4.

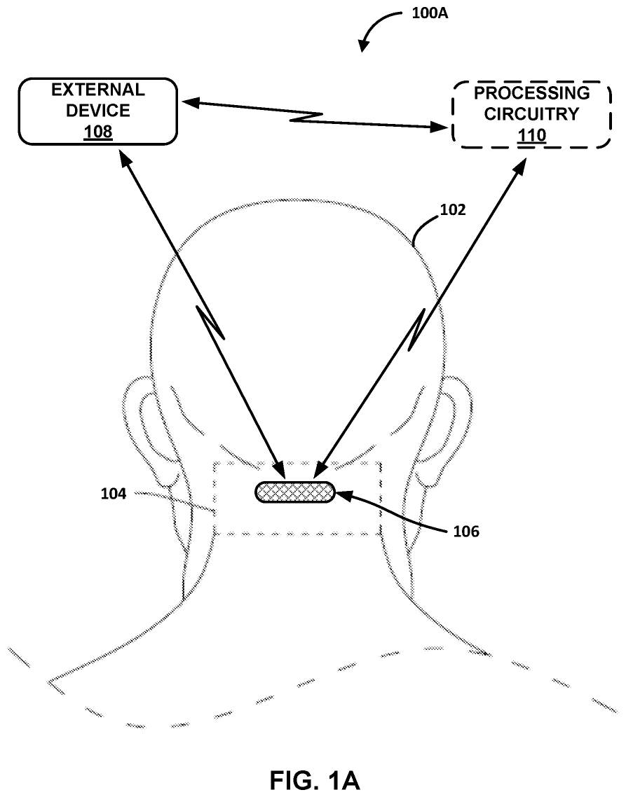

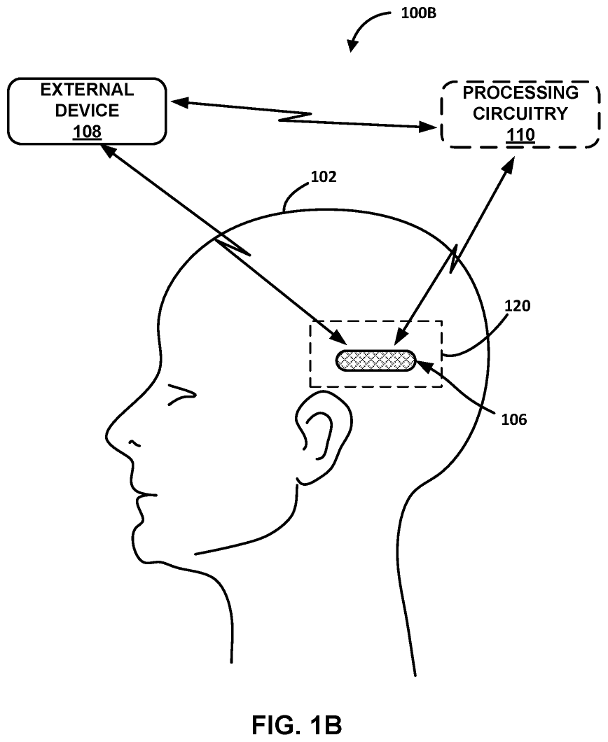

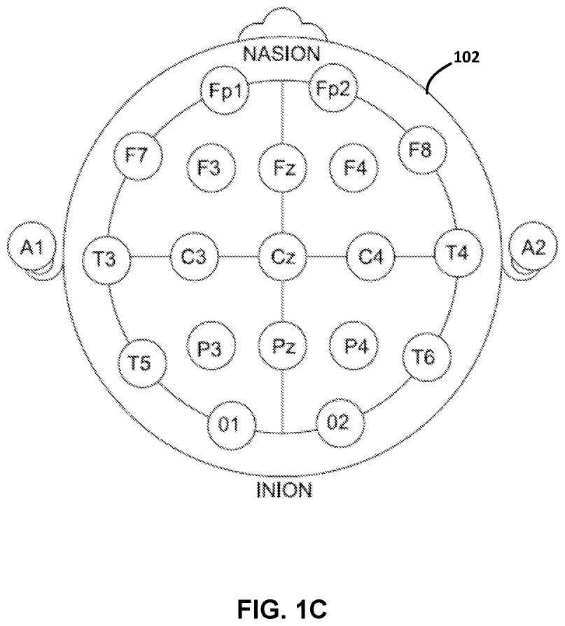

FIG. 6 is a block diagram illustrating an example system that includes an access point, a network, external computing devices, such as a server, and one or more other computing devices, which may be coupled to sensors, the external device, and the processing circuitry of FIG. 1 via a network, in accordance with one or more techniques described herein.

FIG. 7 is a flow diagram illustrating an example of operations for detecting and predicting strokes based on tissue impedance values detected via a plurality of electrodes of sensor devices, in accordance with one or more techniques described herein.

FIG. 8 is a flow diagram illustrating another example of operations for detecting and predicting strokes based on tissue impedance values detected via a plurality of electrodes of sensor devices, in accordance with one or more techniques described herein.

FIG. 9 is a flow diagram illustrating an example of operations for detecting and predicting strokes based on clinical characteristics and tissue impedance values detected via a plurality of electrodes of sensor devices, in accordance with one or more techniques described herein.

FIG. 10 is a flow diagram illustrating an example of operations for generating a stroke threshold based on a normative profile, in accordance with one or techniques described herein.

FIG. 11 is a conceptual diagram of another example system in conjunction with a patient, in accordance with one or more techniques of this disclosure.

Many aspects of the present disclosure can be better understood with reference to the following drawings. The components in the drawings are not necessarily to scale. Instead, emphasis is placed on illustrating clearly the principles of the present technology.

### DETAILED DESCRIPTION

This disclosure describes various systems, devices, and techniques for detecting and predicting stroke from a device coupled with a patient. It can be difficult to determine whether a patient is suffering or will suffer a stroke. Current diagnostic techniques typically involve evaluating a patient for visible symptoms, such as paralysis or numbness of the face, arm, or leg, as well as difficultly walking, speaking, or understanding in the case of stroke. Visible stroke indicators are abbreviated as F.A.S.T.: face, arm, and speech—time to call 9-1-1. However, these techniques may result in undiagnosed strokes, particularly more minor strokes that leave patients relatively functional upon cursory evaluation. Even for relatively minor strokes, it is important to treat the patient as soon as possible because treatment outcomes for stroke patients are highly time-dependent. Accordingly, there is a need for improved methods for detecting and predicting strokes. However, such treatments may be frequently underutilized and/or relatively ineffective due to the failure to timely identify whether a patient is undergoing or has recently undergone a stroke. This is a particular risk with more minor strokes that leave patients relatively functional upon cursory evaluation.

As described herein, a medical device (e.g., an IMD or external medical device wearable by the patient), may be configured to detect and predict the risk of stroke from a location on or near the head of the patient. For example, the IMD may be configured to be implanted subcutaneously without the need for any medical leads. Instead of leads, the IMD may include a housing that carries multiple electrodes directly on the housing. In some examples, however, the IMD may include one or more sensing leads extending therefrom and into the tissue of the patient; such lead(s) may be employed instead of or in addition to the electrodes of the IMD, and may perform any of the functions attributed herein to the electrodes. Using these housing electrodes, the IMD may sense electrical signals and generate tissue impedance values representative of the ejection fraction of the heart of the patient. The IMD may then generate, based on the tissue impedance values representative of ejection fraction of the heart of the patient and other parameters indicative of brain activity, cardiac activity, and/or activity of other organs, a stroke metric indicative of the risk of stroke for the patient. The IMD may output an indication of the detection and/or prediction to a computing device, e.g., to facilitate treatment or intervention.

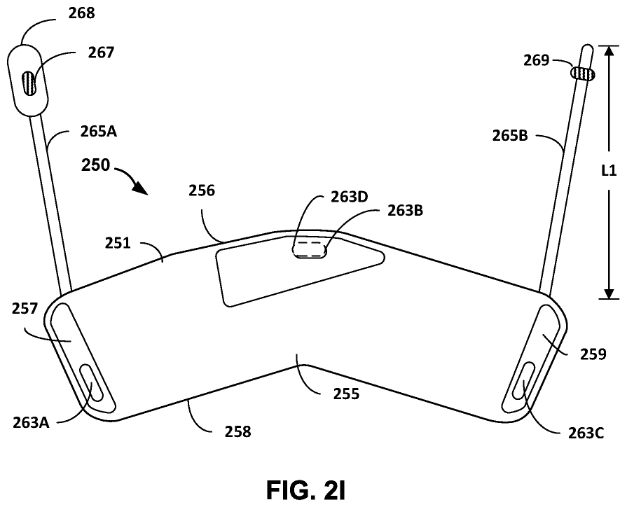

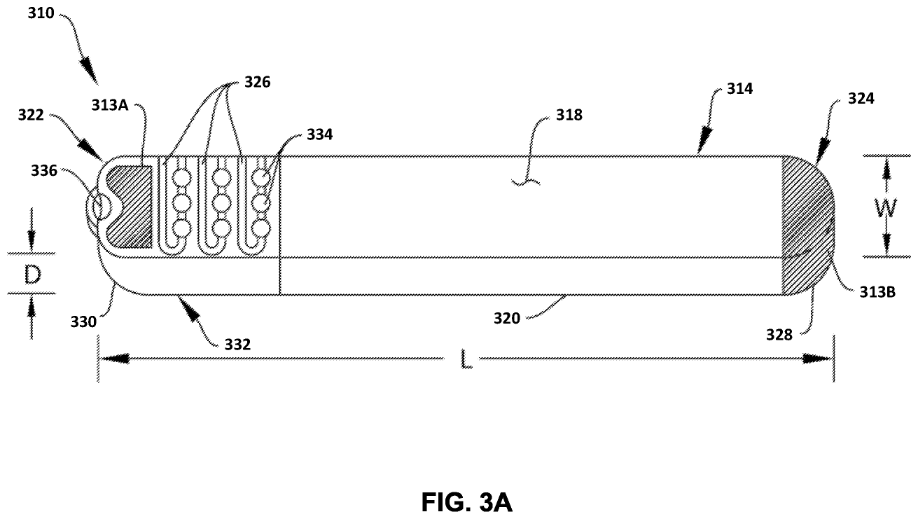

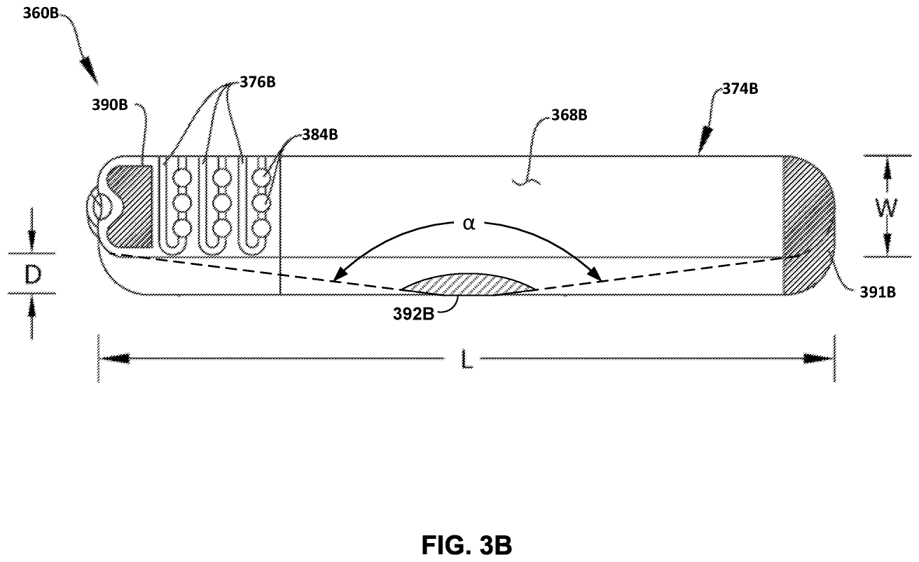

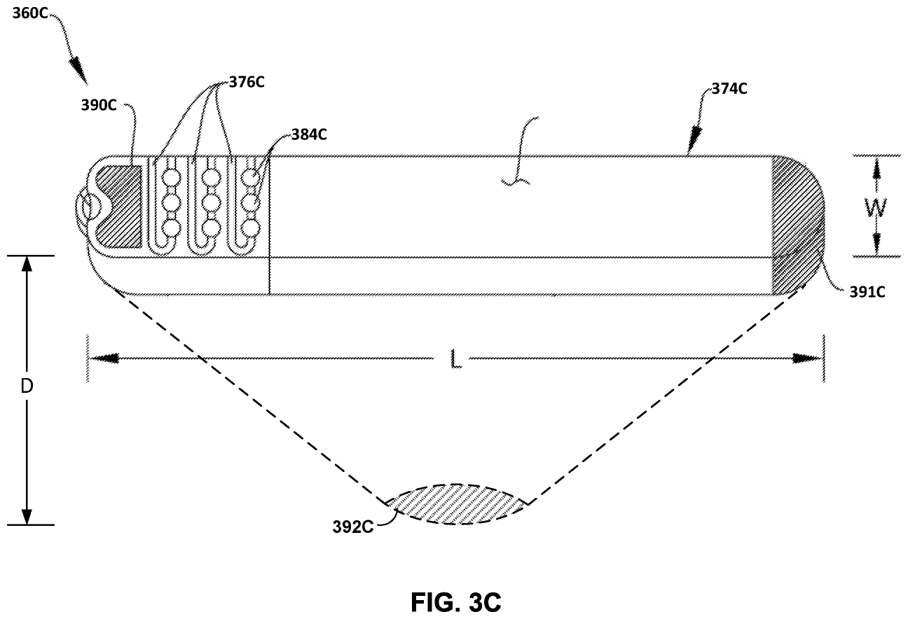

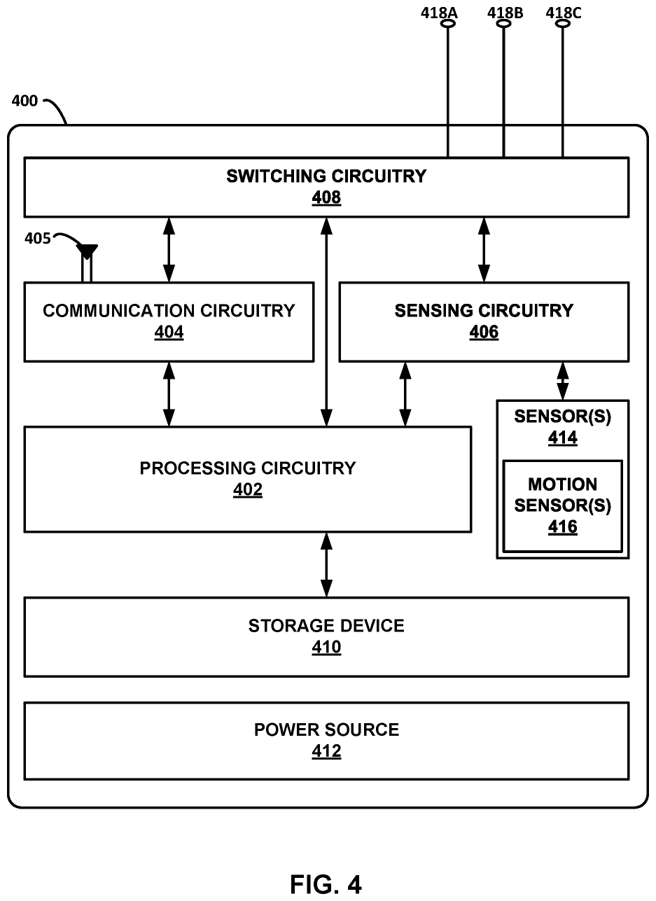

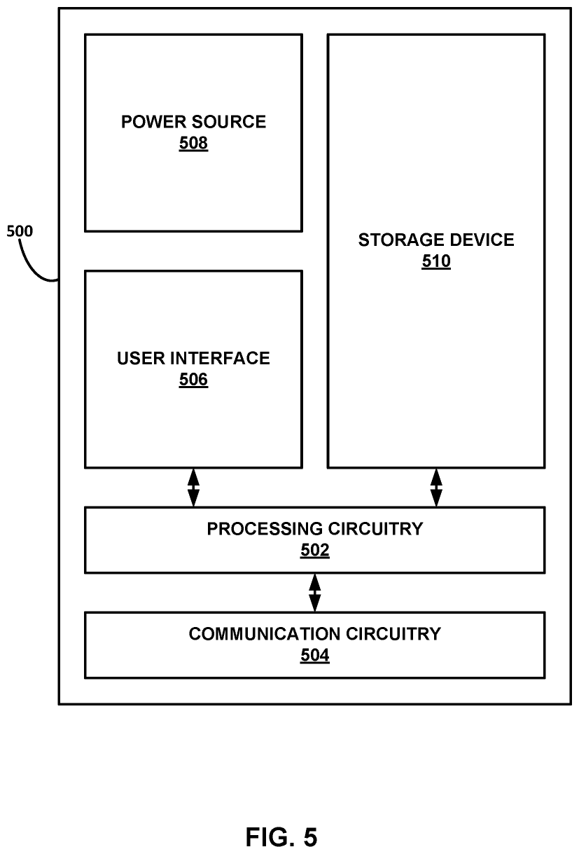

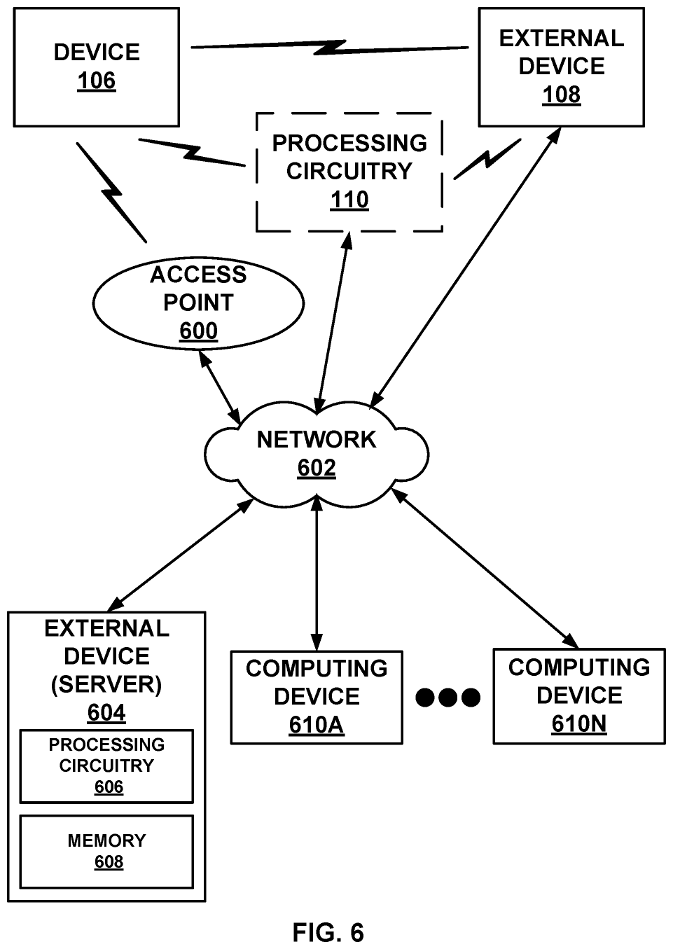

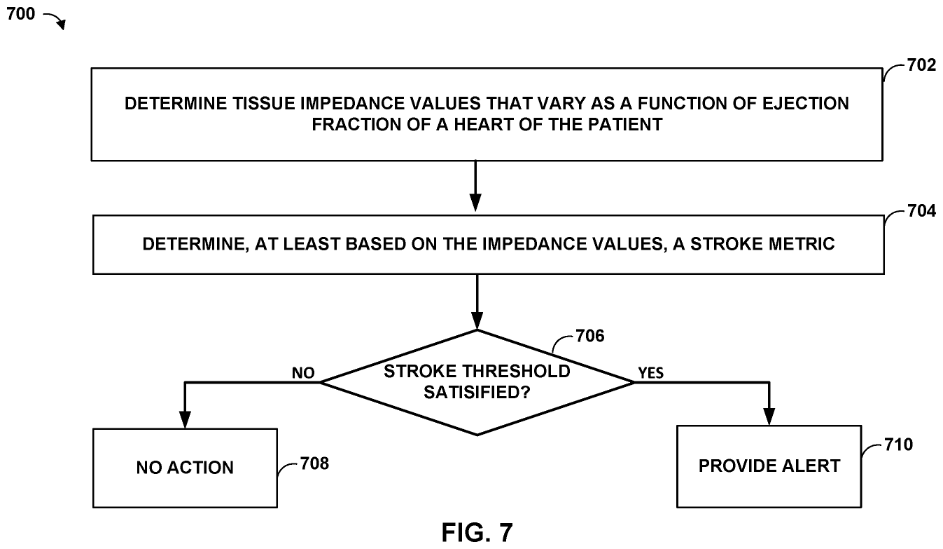

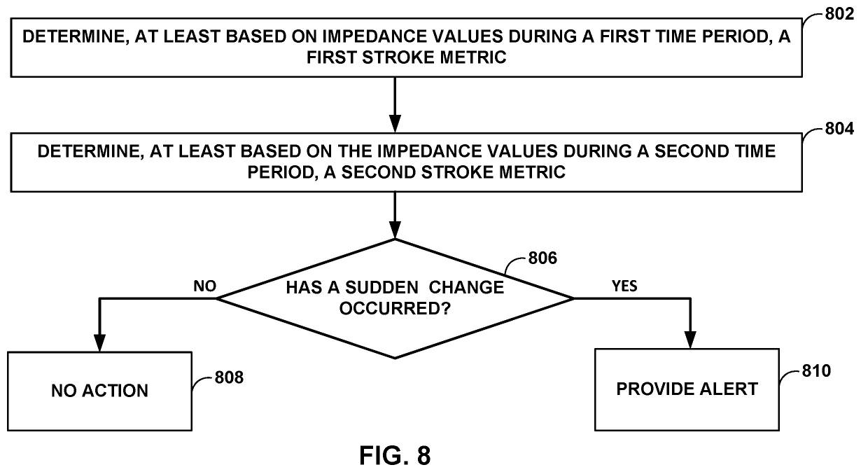

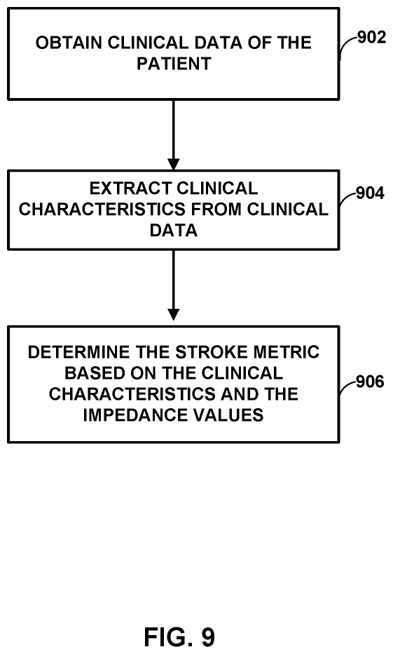

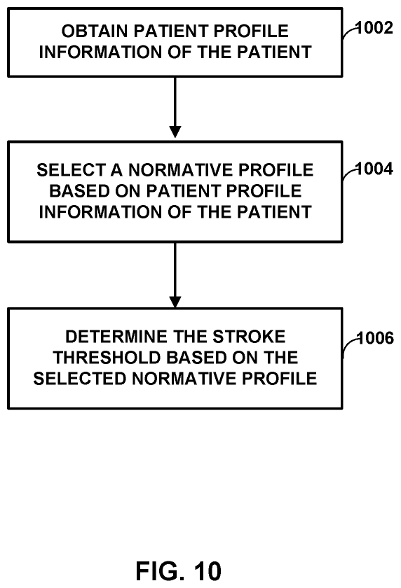

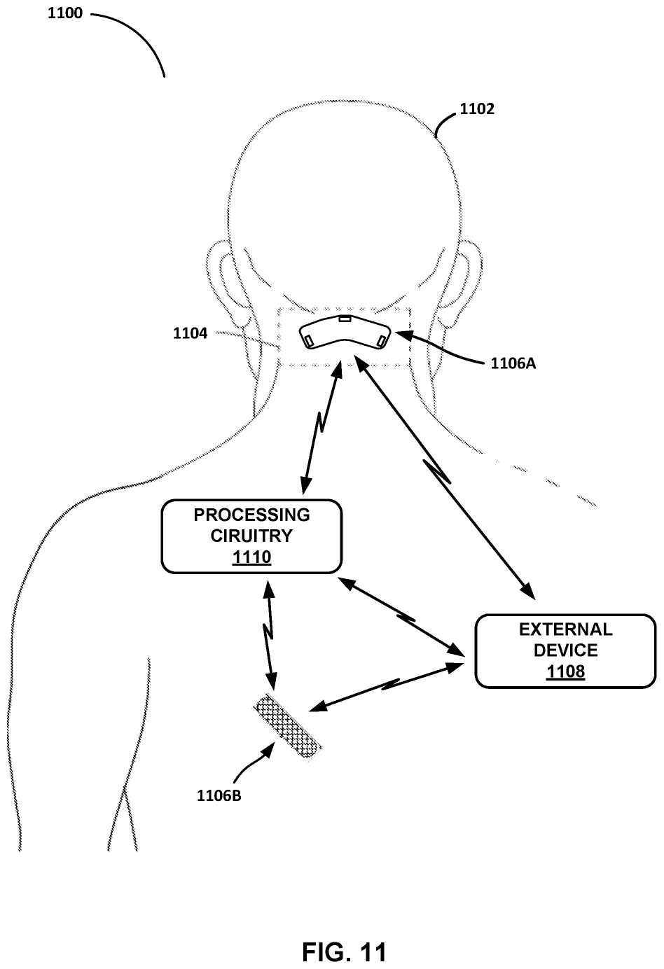

Conventional electroencephalogram (EEG) electrodes are typically positioned over a large portion of a user's scalp. While electrodes in this region are well positioned to detect electrical activity from the patient's brain, there are certain drawbacks. Sensors in this location interfere with patient movement and daily activities, making them impractical for prolonged monitoring. Additionally, implanting traditional electrodes under the patient's scalp is difficult and may lead to significant patient discomfort. To address these and other shortcomings of conventional EEG sensors, sensor devices, according to the technology described herein, sense electrical signals from a smaller region near or on the patient's head, such as adjacent a rear portion of the patient's neck or the base of the patient's skull or near the patient's temple. In these positions, implantation under the patient's skin is relatively simple, and a temporary application of a wearable sensor device (e.g., coupled to a bandage, garment, band, or adhesive member) does not unduly interfere with patient movement and activity. However, in some examples, e.g., as described with respect to FIG. 21, a sensor device may include electrode extensions to increase a size of a vector for sensing impedance signals and/or other electrical signals, such as ECG and EEG signals, which may enhance the sensitivity of stroke detection algorithms using such signals.

The signals detected via electrodes implanted as described herein, e.g., disposed at or adjacent to the back of a patient's neck, may include other signals and relatively high noise amplitude. For example, electrical signals associated with brain activity may be intermixed with electrical signals associated with cardiac activity (e.g., ECG signals) and muscle activity (e.g., electromyogram (EMG) signals) and artifacts from other electrical sources such as patient movement or external interference. Accordingly, in some examples, the signals may be filtered or otherwise manipulated to separate the brain activity data (e.g., EEG signals) and cardiac electrical signals (e.g., ECG signals) from each other and other electrical signals (e.g., EMG signals, etc.). A sensor device of this disclosure may include multiple electrodes having non-parallel vector axes for sensing differential signals, and circuitry in the device may be configured to generate signals, such as an ECG signal and an EEG signal, based on the differential signals.

As described in more detail below, the parameter values may be analyzed to detect or predict stroke based on one or more thresholds or correlation between signals which can itself be derived using machine learning techniques applied to databases patient data known to represent stroke condition. The detection algorithm(s) can be passive (involving measurement of a purely resting patient) or active (involving prompting a patient to perform potentially impaired functionality, such as moving particular muscle groups (e.g., raising an arm, moving a finger, moving facial muscles, etc.,) and/or speaking while recording the electrical response), or from an electrical or other stimulus.

Aspects of the technology described herein can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network (e.g., a wireless communication network, a wired communication network, a cellular communication network, the Internet, a short-range radio network (e.g., via Bluetooth)). In a distributed computing environment, program modules may be located in both local and remote memory storage devices.

Computer-implemented instructions, data structures, screen displays, and other data under aspects of the technology may be stored or distributed on computer-readable storage media, including magnetically or optically readable computer disks, as microcode on semiconductor memory, nanotechnology memory, organic or optical memory, or other portable and/or non-transitory data storage media. In some embodiments, aspects of the technology may be distributed over the Internet or over other networks (e.g., a Bluetooth network) on a propagated signal on a propagation medium (e.g., an electromagnetic wave(s), a sound wave) over a period of time, or may be provided on any analog or digital network (packet switched, circuit switched, or other schemes).

FIG. 1A is a conceptual diagram of a system 100 configured to detect and predict stroke in accordance with examples of the present disclosure. The example techniques described herein may be used with a sensor device 106, which in the illustrated example is an implantable medical device (IMD), and which may be in wireless communication with at least one of external device 108, processing circuitry 110, and other devices not pictured in FIG. 1A. For example, an external device (not illustrated in FIG. 1A) may include at least a portion of processing circuitry 110.

As shown in FIG. 1A, sensor device 106 is located in target region 104. Target region 104 can be outside the thorax, at a rear portion of the neck, or at the base of the skull of patient 102. Although sensor device 106 may be implanted at a location generally centered with respect to the thorax, the head, neck, or target region 104, sensor device 106 may be implanted in an off-center location in order to obtain desired vectors from the electrodes carried on the housing of sensor device 106. Sensor device106 can be disposed in target region 104 either via implantation (e.g., subcutaneously) or by being placed over the patient's skin with one or more electrodes of sensor device 106 being in direct contact with the patient's skin at or adjacent the target region 104.

While conventional EEG electrodes are placed over the patient's scalp and ECG electrodes are positioned elsewhere on the patient's body, the present technology advantageously enables recording of clinically useful brain activity and cardiac activity signals via electrodes positioned at the target region 104 at the rear of the patient's neck or head. This anatomical area is well suited to suited both to implantation of sensor device 106 and to temporary placement of a sensor device over the patient's skin. In contrast, EEG electrodes positioned over the scalp are cumbersome, and implantation over the patient's skull is challenging and may introduce significant patient discomfort.

As noted elsewhere here, conventional EEG electrodes are typically positioned over the scalp to more readily achieve a suitable signal-to-noise ratio for detection of brain activity. However, by using certain digital signal processing, clinically useful brain activity and cardiac activity signals can be obtained using electrodes disposed at the target region 104. Specifically, the electrodes can detect electrical activity that corresponds to brain activity in the P3, Pz, and/or P4 regions (as shown in FIG. 1C).

Processing circuitry 110 may extract values of one or more parameters, e.g., features, from signals indicative of brain activity and/or cardiac activity. Processing circuitry 110 may then determine whether or not the patient has experienced (or has a supra-threshold risk of experiencing) a stroke based on these parameter values. In some examples, sensor device 106 takes the form of a LINQ™ Insertable Cardiac Monitor (ICM), available from Medtronic plc, of Dublin, Ireland, or a device that has a similar implant volume and similar sensing capabilities. The example techniques may additionally, or alternatively, be used with a medical device not illustrated in FIG. 1A such as another type of IMD, a patch monitor device, a wearable device (e.g., smartwatch), or another type of external medical device.

Clinicians sometimes diagnose a patient (e.g., patient 102) with medical conditions (e.g., stroke) and/or determine whether a condition of patient 102 is improving or worsening based on one or more observed physiological signals collected by physiological sensors, such as electrodes, optical sensors, chemical sensors, temperature sensors, acoustic sensors, and motion sensors. In some cases, clinicians apply non-invasive sensors to patients in order to sense one or more physiological signals while a patent is in a clinic for a medical appointment. However, in some examples, events that may change a condition of a patient, such as administration of a therapy, may occur outside of the clinic. As such, in these examples, a clinician may be unable to observe the physiological markers needed to determine whether an event, such as a stroke, has changed a medical condition of the patient and/or determine whether a medical condition of the patient is improving or worsening while monitoring one or more physiological signals of the patient during a medical appointment. In the example illustrated in FIG. 1A, sensor device 106 is implanted within or attached to patient 102 to continuously record one or more physiological signals of patient 102 over an extended period of time.

In some examples, sensor device 106 includes a plurality of electrodes. Sensor device 106 may sense tissue impedance values representative of the ejection fraction of the heart of patient 102. Sensor device 106 may further sense brain electrical activity and heart electrical activity signals, as well as other signals such as impedance signals for respiration, skin impedance, and perfusion, in some examples. Moreover, sensor device 106 may additionally or alternatively include one or more optical sensors, accelerometers or other motion sensors, temperature sensors, chemical sensors, light sensors, pressure sensors, and acoustic sensors, in some examples. Such sensors may sense various signals that may improve the ability of processing circuitry 110 to detect and/or predict stroke.

External device 108 may be a hand-held computing device with a display viewable by the user and an interface for providing input to external device 108 (e.g., a user input mechanism). For example, external device 108 may include a small display screen (e.g., a liquid crystal display (LCD) or a light emitting diode (LED) display) that presents information to the user. In addition, external device 108 may include a touch screen display, keypad, buttons, a peripheral pointing device, voice activation, or another input mechanism that allows the user to navigate through the user interface of external device 108 and provide input. If external device 108 includes buttons and a keypad, the buttons may be dedicated to performing a certain function, e.g., a power button, the buttons and the keypad may be soft keys that change in function depending upon the section of the user interface currently viewed by the user, or any combination thereof. In some examples, external device 108 is a smartphone of patient 102, which may communicate with sensor device 106, e.g., via Bluetooth™.

In other examples, external device 108 may be a larger workstation or a separate application within another multi-function device, rather than a dedicated computing device. For example, the multi-function device may be a notebook computer, tablet computer, workstation, one or more servers, cellular phone, personal digital assistant, or another computing device that may run an application that enables the computing device to operate as a secure device. In some examples, external device 108 is configured to communicate with a computer network, such as the Medtronic CareLink® Network developed by Medtronic, plc, of Dublin, Ireland.

Processing circuitry 110, in some examples, may include one or more processors that are configured to implement functionality and/or process instructions for execution within IMD 106. For example, processing circuitry 110 may be capable of processing instructions stored in a storage device. Processing circuitry 110 may include, for example, microprocessors, digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 110 may include any suitable structure, whether in hardware, software, firmware, or any combination thereof, to perform the functions ascribed herein to processing circuitry 110.

Processing circuitry 110 may represent processing circuitry located within any one or both of sensor device 106 and external device 108. In some examples, processing circuitry 110 may be entirely located within a housing of sensor device 106. In other examples, processing circuitry 110 may be entirely located within a housing of external device 108. In other examples, processing circuitry 110 may be located within any one or combination of sensor device 106, external device 108, and another device or group of devices that are not illustrated in FIG. 1A. As such, techniques and capabilities attributed herein to processing circuitry 110 may be attributed to any combination of sensor device 106, external device 108, and other devices that are not illustrated in FIG. 1A.

Medical device system 100A of FIG. 1A is an example of a system configured to sense signals and detect and predict the risk of stroke of patient 102 according to one or more techniques of this disclosure. In some examples, the sensed signals may include a plurality of tissue impedance values that vary as a function of ejection fraction of the heart of patient 102. Processing circuitry 110 may determine a stroke metric indicative of a stroke status of patient 102 based on the plurality of tissue impedance values, e.g., alone or in combination with the other parameters described herein. Processing circuitry 110 may further store the stoke metric in memory of medical device system 100A.

In some examples, the sensed signals may include other features representative of heart function such as depolarizations and repolarizations of the heart. Processing circuitry 110 may perform signal processing techniques to extract information indicating the one or more parameters of the cardiac signal. In other some examples, the sensed electrical signals may include features representative of brain function, such as amplitudes of frequencies in one or more frequency bands, such as alpha bands, beta bands, or gamma bands. Processing circuitry 110 may perform various signal processing techniques to extract these brain features from the sensed electrical signals.

In some examples, sensor device 106 includes one or more accelerometers or other motion sensors. An accelerometer of sensor device 106 may collect an accelerometer signal, which reflects a measurement of any one or more of a motion of patient 102, a posture of patient 102 and a facial expression of patient 102. In some cases, the accelerometer may collect a three-axis accelerometer signal indicative of patient 102's movements within a three-dimensional Cartesian space. For example, the accelerometer signal may include a vertical axis accelerometer signal vector, a lateral axis accelerometer signal vector, and a frontal axis accelerometer signal vector. The vertical axis accelerometer signal vector may represent an acceleration of patient 102 along a vertical axis, the lateral axis accelerometer signal vector may represent an acceleration of patient 102 along a lateral axis, and the frontal axis accelerometer signal vector may represent an acceleration of patient 102 along a frontal axis. In some cases, the vertical axis substantially extends along a torso of patient 102 when patient 102 from a neck of patient 102 to a waist of patient 102, the lateral axis extends across a chest of patient 102 perpendicular to the vertical axis, and the frontal axis extends outward from and through the chest of patient 102, the frontal axis being perpendicular to the vertical axis and the lateral axis.

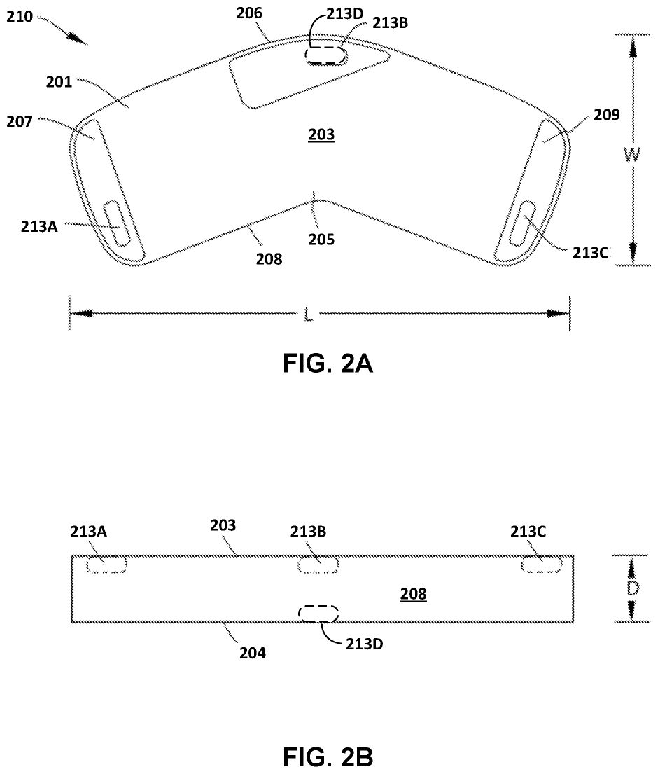

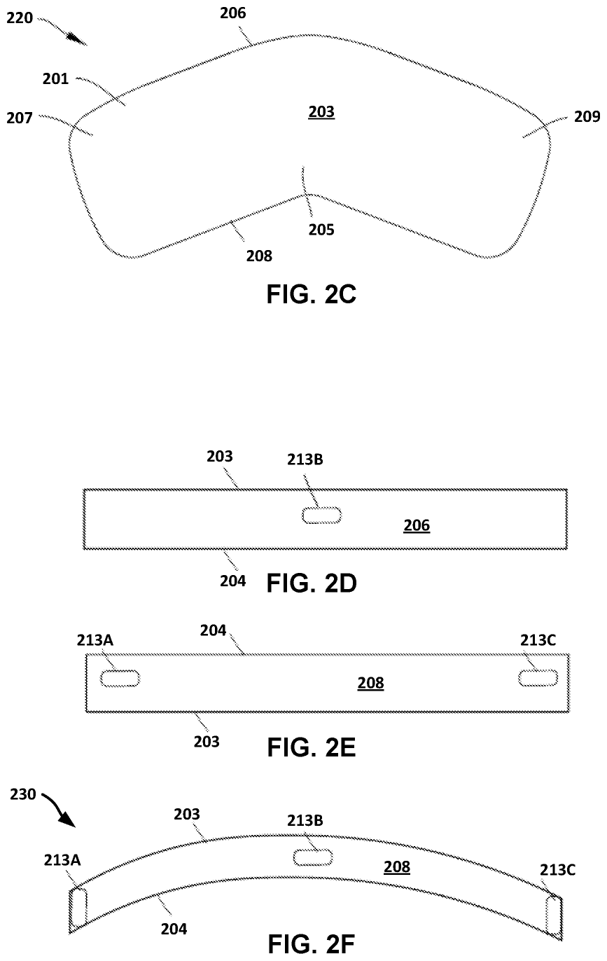

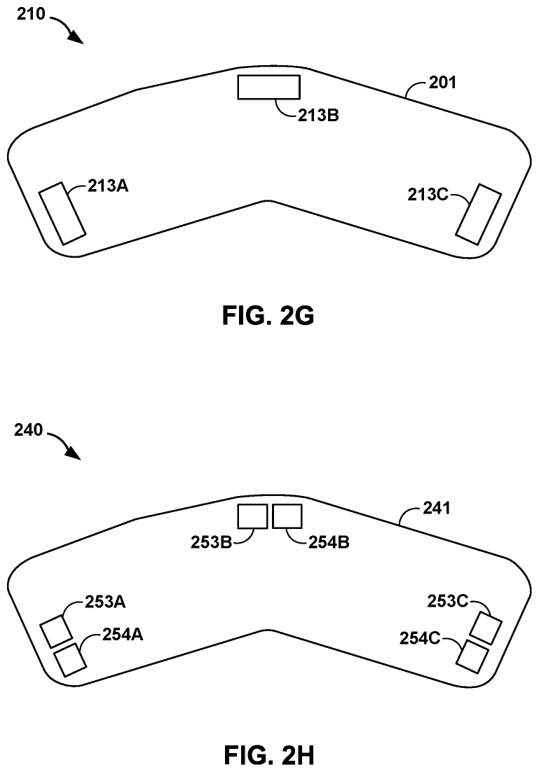

Sensor device 106 may measure other signals, such as an impedance (e.g., subcutaneous impedance measured via electrode depicted in FIGS. 2A-2I), which may indicate respiration, skin impedance, or perfusion, ejection fraction, or other cardiac performance parameters. Additional signals may include heart sound signals, ballistocardiogram signals, pressure signals, or the like. Processing circuitry 110 may analyze any one or more of the set of parameters in order to determine whether or not patient 102 is experiencing or has a supra-threshold risk of experiencing a stroke.

In some examples, one or more sensors (e.g., electrodes, motion sensors, optical sensors, temperature sensors, pressure sensors, or any combination thereof) of sensor device 106 may generate a signal that indicates a parameter of a patient. In some examples, the signal that indicates the parameter includes a plurality of parameter values, where each parameter value of the plurality of parameter values represents a measurement of the parameter at a respective interval of time. The plurality of parameter values may represent a sequence of parameter values over time, where each parameter value of the sequence of parameter values are collected by sensor device 106 for each time interval of a sequence of time intervals. For example, sensor device 106 may perform a parameter measurement in order to determine a parameter value of the sequence of parameter values according to a recurring time interval (e.g., every day, every night, every other day, every twelve hours, every hour, every second, or any other recurring time interval). In this way, sensor device 106 may be configured to track a respective patient parameter more effectively as compared with a technique in which a patient parameter is tracked during patient visits to a clinic, since IMD 106 is implanted within patient 102 and is configured to perform parameter measurements according to recurring time intervals without missing a time interval or performing a parameter measurement off schedule.

Sensor device 106 may be referred to as a system or device. In one example, sensor device 106 may include a plurality of electrodes carried by the housing of sensor device 106, sensing circuitry configured to sense, via at least two electrodes of the plurality of electrodes, electrical signals from patient 10, and a motions sensor, e.g., accelerometer, configured to sense a motion signal of patient 10. Sensor device 106 may also include processing circuitry 110. The housing of sensor device 106 carries the plurality of electrodes and contains, or houses, the sensing circuitry, the processing circuitry, the motion sensor, and any other sensors. In this manner, sensor device 106 may be referred to as a leadless sensing device because the electrodes are carried directly by the housing instead of by any leads that extend from the housing. In some examples, however, sensor device 106 may include one or more sensing leads extending therefrom and into the tissue of the patient; such lead(s) may be employed instead of or in addition to the electrodes of sensor device 106 (e.g., such as electrode extensions depicted in FIG. 2I), and may perform any of the functions attributed herein to the electrodes.

The signals sensed by sensing device 106 can include electrical brain signals and/or electrical heart signals. In some examples, the plurality of electrodes are configured to detect brain signals corresponding to activity in at least one of a P3, Pz, or P4 brain region, which is at the back of the head or upper neck region as shown in FIG. 1C. In this manner, the housing of sensor device 106 may be configured to be disposed at or adjacent to a rear portion of a neck or skull base of patient 102. The housing of sensor device 106 may be configured to be implanted within patient 102, such as implanted subcutaneously. In other examples, the housing of sensor device 106 may be configured to be disposed on an external surface of the skin of patient 102.

In some examples, sensor device 106 may include a single sensing circuitry configured to generate, from the sensed electrical signals, information that includes both the electrical brain activity data (e.g., electroencephalogram (EEG) data) and the electrical heart activity data (e.g., electrocardiogram (ECG) data). In other examples, the processing circuity of sensor device 106 may include separate hardware that generates different information from the sensed electrical signals. For example, IMD 106 may include first circuitry configured to generate the electrical brain activity from the electrical signals and second circuitry different from the first circuitry and configured to generate the electrical heart activity data from the electrical signals. Even with the first and second circuitry configured to generate different information, or data, in some examples, sensed electrical signals may be conditioned or processed by one or more electrical components (e.g., filters or amplifiers) prior to being processed by the first and second circuitry. In some examples, parameters determined from electrical brain activity signals data may include features, such as spectral features, indicative of the strength of signals in various frequency bands or at various frequencies.

In some examples, sensor device 106 may include one or more accelerometers or other motion sensors within the housing. The accelerometer may be configured to generate motion data representative of the motion of patient 102. Processing circuitry 110 may then be configured to generate the detection or prediction of stroke based on the motion signal, e.g., in combination with the parameter values determined from the brain and cardiac signals. For example, certain body motions or behaviors (e.g., patterns of motion) may be indicative of stroke experienced by patient 102. In one example, the processing circuitry 110 may be configured to determine, based on the motion data, that patient 102 has fallen, or has nearly fallen. In response to determining that patient 102 has fallen, the processing circuitry 110 may be configured to inform or modify an algorithm for detecting or predicting stroke. In some examples, a stroke may cause a patient to fall. Therefore, in combination with other features extracted from sensed electrical signals, processing circuitry 110 may determine from the fall indication that the stroke metric indicates detection of a stroke. In other examples, sensor device 106 or processing circuitry 110 may determine that a characteristic of the motion data exceeds a threshold. The threshold may be an acceleration value indicative of a fall, for example.

FIG. 1B is a conceptual diagram of a system 100B configured to detect and predict stroke of patient 102 in accordance with examples of the present disclosure. System 100B may be substantially similar to system 100A of FIG. 1A. However, sensor device 106 of system 100B may be configured to be implanted in target region 120, which is located on the side of the head posterior of the temple of patient 102. Sensor device 106 implanted at target region 120 may be configured to sense cardiac electrical and brain electrical signals, as well as other sensor signals described herein, in this area. In some examples, sensor device 106 may need to employ different filters or other processing or signal conditioning techniques than those at target region 104 due to different types of noise at target region 120, such as muscle activity due to mandible movement or other types of electrical activity. In other examples, sensor device 106 may be configured to sense signals as described herein from other areas of the head of patient 102 that may be outside of target regions 104 and 120.

FIG. 1C is a diagram of the 10-20 map for electroencephalography (EEG) sensor measurements. As shown in FIG. 1C, various locations on the head of patient 102 may be targeted using the electrodes carried by sensor device 106. At the back of the head, such as in target region 104 of FIG. 1A, sensor device 106 may sense electrical signals at least one of P3, Pz or P4. At the side of the head, such as in target region 120 of FIG. 1B, sensor device 106 may sense electrical signals at least one of F7, T3, or T5 and/or at one or more of F8, T4, or T6.

FIG. 2A depicts a top view of a sensor device 210 (e.g., an IMD) in accordance with examples of this disclosure. FIG. 2B depicts a side view of sensor device 210 shown in FIG. 2A. In some examples, sensor device 210 can include some or all of the features of, and be similar to, sensor device 106 described above with respect to FIGS. 1A and 1B and/or the sensor devices 310, 360B, 360B, or 400 described below with respect to FIGS. 3A-3C and 4, and can include additional features as described in connection with FIG. 2A. In the illustrated example, sensor device 210 includes a housing 201 that carries a plurality of electrodes 213A, 213B,213C, and 213D (collectively “electrodes 213”) therein. Although four electrodes are shown for sensor device 210, in other examples, only two or three electrodes may be carried by housing 201, e.g., on a common surface of housing 203. As shown in FIG. 2H, any of the electrodes may be segmented; that is, each electrode may include two conductive portions separated by an insulative material. In some examples, a first portion may be configured to sense ECG signals, and a second portion may be configured to sense EEG signals.

In operation, electrodes 213 can be placed in direct contact with tissue at the target site (e.g., with the user's skin if placed over the user's skin, or with subcutaneous tissue if the sensor device 210 is implanted). Housing 201 additionally encloses electronic circuitry located inside the sensor device 210 and protects the circuitry (e.g., processing circuitry, sensing circuitry, communication circuitry, sensors, and a power source) contained therein from body fluids. In various examples, electrodes 213 can be disposed along any surface of the sensor device 210 (e.g., anterior surface, posterior surface, left lateral surface, right lateral surface, superior side surface, inferior side surface, or otherwise), and the surface, in turn, may take any suitable form.

In the example of FIGS. 2A and 2B, housing 201 can be a biocompatible material having a relatively planar shape including a first major surface 203 configured to face towards the tissue of interest (e.g., to face anteriorly when positioned at the back of the patient's neck) a second major surface 204 opposite the first, and a depth D or thickness of housing 201 extending between the first and second major surfaces. Housing 201 can define a superior side surface 206 (e.g., configured to face superiorly when sensing device 210 is implanted in or at the patient's head or neck) and an opposing inferior side surface 208. Housing 201 can further include a central portion 205, a first lateral portion (or left portion) 207, and a second lateral portion (or right portion) 209. Electrodes 213 are distributed about housing 201 such that a central electrode 213B is disposed within the central portion 205 (e.g., substantially centrally along a horizontal axis of the device), a back electrode 213D is disposed on inferior side surface, a left electrode 213A is disposed within the left portion 207, and a right electrode 213C is disposed within the right portion 209. As illustrated, housing 201 can define a boomerang or chevron-like shape in which the central portion 205 includes a vertex, with the first and second lateral portions 207 and 209 extending both laterally outward and from the central portion 205 and also at a downward angle with respect to a horizontal axis of the device. In other examples, housing 201 may be formed in other shapes, which may be determined by desired distances or angles between different electrodes 213 carried by housing 201.

The configuration of housing 201 can facilitate placement either over the user's skin in a wearable or bandage-like form or for subcutaneous implantation. As such, a relatively thin housing 201 can be advantageous. Additionally, housing 201 can be flexible in some embodiments, so that housing 201 can at least partially bend to correspond to the anatomy of the patient's neck (e.g., with left and right lateral portions 207 and 209 of housing 201 bending anteriorly relative to the central portion 205 of housing 201).

In some embodiments, housing 201 can have a length L of from about 15 to about 50 mm, from about 20 to about 30 mm, or about 25 mm. Housing 201 can have a width W from about 2.5 to about 15 mm, from about 5 to about 10 mm, or about 7.5 mm. In some embodiments, housing 201 can have a thickness of the thickness is less than about 10 mm, about 9 mm, about 8 mm, about 7 mm, about 6 mm, about 5 mm, about 4 mm, or about 3 mm. In some embodiments, the thickness of housing 201 can be from about 2 to about 8 mm, from about 3 to about 5 mm, or about 4 mm. Housing 201 can have a volume of less than about 1.5 cc, about 1.4 cc, about 1.3 cc, about 1.2 cc, about 1.1 cc, about 1.0 cc, about 0.9 cc, about 0.8 cc, about 0.7 cc, about 0.6 cc, about 0.5 cc, or about 0.4 cc. In some embodiments, housing 201 can have dimensions suitable for implantation through a trocar introducer or any other suitable implantation technique.

As illustrated, electrodes 213 carried by housing 201 are arranged so that the electrodes 213 do not lie on a common axis. In such a configuration, electrodes 213 can achieve a better signal vector as compared to electrodes that are all aligned along a single axis. This can be particularly useful in a sensor device 210 configured to be implanted at the neck or head while detecting electrical activity in the brain and the heart.

In some examples, all electrodes 213 are located on the first major surface 203 and are substantially flat and outwardly facing. However, in other examples, one or more electrodes 213 may utilize a three-dimensional configuration (e.g., curved around an edge of the device 210). Similarly, in other examples, such as that illustrated in FIG. 2B, one or more electrodes 213 may be disposed on the second major surface opposite the first. The various electrode configurations allow for configurations in which electrodes 213 are located on both the first major surface and the second major surface. Electrodes 213 may be formed of a plurality of different types of biocompatible conductive material (e.g., stainless steel, titanium, platinum, iridium, or alloys thereof), and may utilize one or more coatings such as titanium nitride or fractal titanium nitride. In some examples, the material choice for electrodes can also include materials having a high surface area (e.g., to provide better electrode capacitance for better sensitivity) and roughness (e.g., to aid implant stability). Although the example shown in FIGS. 2A and 2B includes four electrodes 213, in some embodiments, the sensor device 210 can include 1, 2, 3, 5, 6, or more electrodes carried by housing 201.

FIG. 2C depicts a top view of another example sensor device 220 in accordance with the present technology. FIG. 2C illustrates sensor device 220, which is substantially similar to sensor device 210, but sensor device 220 includes electrodes 213, which are not exposed along the first major surface 203 of housing 201. Instead, electrodes 213 can be exposed along superior and inferior side surfaces (e.g., facing superiorly and inferiorly when implanted at or on a patient's neck), as shown in FIGS. 2D and 2E. FIG. 2F illustrates sensor device 230, which is substantially similar to sensor devices 210 and 220, but housing 201 is constructed to have a curved configuration, and in which the electrodes can be placed along the superior and/or inferior side surfaces of housing 201. In some embodiments, a curved configuration can improve patient comfort and more readily conform to the anatomy of the patient's neck region. In some examples, any of sensor devices 210, 220, or 230 may be flexible in order to conform to the anatomy of the patient at the desired implant or external surface location. Additionally, examples that include electrode extensions, e.g., as depicted in FIG. 2I are inherently flexible, allowing conformance to neck and/or cranial anatomy. In some examples, sensor device 220 and/or sensor device 230 may be implanted at a location generally centered with respect to the thorax, the head, neck, or a target region. In some examples, sensor device 220 and/or sensor device 230 may be placed on an external surface of skin of a patient.

In operation, electrodes 213 are used to sense electrical signals (e.g., EEG or other brain electrical signals and/or ECG or other heart electrical signals) which may be submuscular or subcutaneous. Electrodes 213 may also be used to sense impedance of tissue proximate to the electrodes. The sensed electrical signals may be stored in a memory of the sensor device, and data may be transmitted via a communications link to another device (e.g., external device 108 of FIG. 1A). The signals may be time-coded or otherwise correlated with time data, and stored in this form, so that the recency, frequency, time of day, time span, or date(s) of a particular signal data point or data series (or computed measures or statistics based thereon) may be determined and/or reported. In some examples, electrodes 213 may additionally or alternatively be used for sensing any bio-potential signal of interest, such as electromyogram (EMG) or a nerve signal, as well as impedance signals, from any implanted or external location. These signals may be time-coded or time-correlated, and stored in that form, in the manner described above with respect to brain and cardiac signal data.

FIGS. 2G and 2H depict top views of devices in accordance with examples of the present disclosure. FIG. 2G depicts housing 201 of sensor device 210, which includes electrodes 213A-213C arranged at the perimeter of housing 201. Each of electrodes 213A-213C may be configured to receive raw signals including ECG and EEG components. Sensor device 210 may include circuitry configured to filter the raw signals received by electrodes 213A-213C to generate ECG signals and EEG signals. Sensor device 210 may also include circuitry configured to measure impedance of tissue via electrodes 213A-213C. In some examples, this circuitry may be located outside of sensor device 210.

FIG. 2H depicts housing 241 of sensor device 240, which includes electrodes 253A-253C and 254A-254C. Electrodes 253A and 254A together may be referred to as a segmented electrode. Similarly, electrodes 253B and 254B may be referred to as a segmented electrode, and electrodes 253C and 254C may be referred to as a segmented electrode. Insulative material may separate the conductive portions (e.g., electrodes 253A and 254A) of a segmented electrode.

Circuitry may be configured to generate a first ECG signal based on a differential signal received at electrodes 253A and 253B, generate a second ECG signal based on a differential signal received at electrodes 253B and 253C, and/or generate a third ECG signal based on a differential signal received at electrodes 253C and 253A. Likewise, the circuitry may be configured to generate a first EEG signal based on a differential signal received at electrodes 254A and 254B, generate a second EEG signal based on a differential signal received at electrodes 254B and 254C, and/or generate a third EEG signal based on a differential signal received at electrodes 254C and 254A.

FIG. 2I depicts a top view of another example sensor device 250, which includes electrodes 263A-236D, 267, and 269. Each of electrodes 263A-236D, 267, and 269 may be configured to receive raw signals including ECG and EEG components. Sensor device 250 may include circuitry configured to filter the raw signals received by electrodes 263A-236D, 267, and 269 to generate ECG signals and EEG signals. Sensor device 250 may also include circuitry configured to measure impedance of tissue via electrodes 263A-236D, 267, and 269.

In the example of FIG. 2I, sensor device 250 include a housing 251,which includes a superior side surface 256, an opposing inferior side surface 258, a central portion 255, a first lateral portion (or left portion) 257, and a second lateral portion (or right portion) 259. Electrodes 263 are distributed about housing 251 such that a central electrode 263B is disposed within the central portion 255 (e.g., substantially centrally along a horizontal axis of the device), a back electrode 263D is disposed on inferior side surface, a left electrode 263A is disposed within the left portion 257, and a right electrode 263C is disposed within the right portion 259.

Sensor device 250 further include electrode extensions 265A and 265B (collectively “electrode extensions 265”). As illustrated in FIG. 2I, electrode extension 265A includes a paddle 268 such that one or more electrodes 267 are distributed on paddle 268. Electrode extension 265B includes one or more ring electrodes 269. In some examples, electrode extensions 265 may be connect to a housing 256 of sensor device 250 via header pins. In some examples, electrode extensions 265 may be permanently attached to housing 256 of sensor device 250.

In some examples, electrode extensions 265 can have a length L1 of from about 15 to about 50 mm, from about 20 to about 30 mm, or about 25 mm. Electrode extensions 265 are inherently flexible, allowing conformance to neck and/or cranial anatomy. Additionally, the configuration of electrode extensions 265 increases a size of a sensing vector for measuring impedance or sensing EEG, ECG, or other electrical signals.

FIGS. 3A-3C depict other example sensor devices 310and 360B in accordance with embodiments of the present technology. In some examples, sensor device 310 can include some or all of the features of IMDs 106 or 400, sensor devices 210, 220, and 230, described herein in accordance with embodiments of the present technology, and can include additional features as described in connection with FIG. 3A. In the example shown in FIG. 3A, sensor device 310 may be embodied as a monitoring device having housing 314, proximal electrode 313A and distal electrode 313B (individually or collectively “electrode 313” or “electrodes 313”). Housing 314 may further comprise first major surface 318, second major surface 320, proximal end 322, and distal end 324. Housing 314 encloses electronic circuitry located inside sensor device 310 and protects the circuitry contained therein from body fluids. Electrical feedthroughs provide electrical connection of electrodes 313. In an example, sensor device 310 may be embodied as an external monitor, such as a patch that may be positioned on an external surface of the patient, or another type of medical device (e.g., instead of as an ICM), such as described further herein.

In the example shown in FIG. 3A, sensor device 310 is defined by a length “L,” a width “W,” and thickness or depth “D.” sensor device 310 may be in the form of an elongated rectangular prism wherein the length L is significantly larger than the width W, which in turn is larger than the depth D. In one example, the geometry of sensor device 310—in particular, a width W being greater than the depth D—is selected to allow sensor device 310 to be inserted under the skin of the patient using a minimally invasive procedure and to remain in the desired orientation during insertion. For example, the device shown in FIG. 3A includes radial asymmetries (notably, the rectangular shape) along the longitudinal axis that maintains the device in the proper orientation following insertion. For example, the spacing between proximal electrode 313a and distal electrode 313B may range from 30 millimeters (mm) to 55 mm, 35 mm to 55 mm, and from 40 mm to 55 mm, and may be any range or individual spacing from 25 mm to 60 mm. In some examples, the length L may be from 30 mm to about 70 mm. In other examples, the length L may range from 40 mm to 60 mm, 45 mm to 60 mm and may be any length or range of lengths between about 30 mm and about 70 mm. In addition, the width W of first major surface 18 may range from 3 mm to 10 mm and may be any single or range of widths between 3 mm and 10 mm. The thickness of depth D of sensor device 310 may range from 2 mm to 9 mm. In other examples, the depth D of sensor device 310 may range from 2 mm to 5 mm and may be any single or range of depths from 2 mm to 9 mm. In addition, sensor device 310, according to an example of the present disclosure, has a geometry and size designed for ease of implant and patient comfort. Examples of sensor device 310 described in this disclosure may have a volume of 3 cc or less, 2 cc or less, 1 cc or less, 0.9 cc or less, 0.8 cc or less, 0.7 cc or less, 0.6 cc or less, 0.5 cc or less, or 0.4 cc or less, any volume between 3 and 0.4 cc. In addition, in the example shown in FIG. 3A, proximal end 322 and distal end 324 are rounded to reduce discomfort and irritation to surrounding tissue once inserted under the skin of the patient. In some examples, sensor device 310 may be implanted at a location generally centered with respect to the thorax, the head, neck, or a target region. In some examples, sensor device 310 may be placed on an external surface of skin of a patient. In some examples, more than one sensor devices may be used to sense signals from the patient.

In the example shown in FIG. 3A, once inserted within the patient, the first major surface 318 faces outward, toward the skin of the patient while the second major surface 320 is located opposite the first major surface 318. Consequently, the first and second major surfaces may face in directions along a sagittal axis of the patient, and this orientation may be consistently achieved upon implantation due to the dimensions of sensor device 310. Additionally, an accelerometer, or axis of an accelerometer, may be oriented along the sagittal axis.

Proximal electrode 313A and distal electrode 313B are used to sense electrical signals (e.g., EEG signals or ECG signals), which may be submuscular or subcutaneous, as well as measure tissue impedances. Electrical signals and impedances may be stored in a memory of sensor device 310, and data may be transmitted via integrated antenna 326 to another medical device, which may be another implantable device or an external device, such as external device 108 (FIG. 1A). In some examples, electrodes 313A and 313B may additionally or alternatively be used for sensing any bio-potential signal of interest, such as an electrocardiogram (ECG), intracardiac electrogram (EGM), electromyogram (EMG), or a nerve signal, from any implanted location.

In the example shown in FIG. 3A, proximal electrode 313A is in close proximity to the proximal end 322, and distal electrode 313B is in close proximity to distal end 324. In this example, distal electrode 313B is not limited to a flattened, outward facing surface, but may extend from first major surface 318 around rounded edges 328 or end surface 330 and onto the second major surface 320 so that the electrode 313B has a three-dimensional curved configuration. In the example shown in FIG. 3A, proximal electrode 313A is located on first major surface 318 and is substantially flat, outward facing. However, in other examples, proximal electrode 313A may utilize the three-dimensional curved configuration of distal electrode 313B, providing a three-dimensional proximal electrode (not shown in this example). Similarly, in other examples, distal electrode 313B may utilize a substantially flat, outward facing electrode located on first major surface 318, similar to that shown with respect to proximal electrode 313A. The various electrode configurations allow for configurations in which proximal electrode 313A and distal electrode 313B are located on both first major surface 318 and second major surface 320. In other configurations, such as that shown in FIG. 3A, only one of proximal electrode 313A and distal electrode 313B is located on both major surfaces 318 and 320, and in still other configurations both proximal electrode 313A and distal electrode 313B are located on one of the first major surface 318 or the second major surface 320 (e.g., proximal electrode 313A located on first major surface 318 while distal electrode 313B is located on second major surface 320). In another example, sensor device 310 may include electrodes 313 on both first major surface 318 and second major surface 320 at or near the proximal and distal ends of the device, such that a total of four electrodes 313 are included on sensor device 310. Electrodes 313 may be formed of a plurality of different types of biocompatible conductive material (e.g., stainless steel, titanium, platinum, iridium, or alloys thereof), and may utilize one or more coatings such as titanium nitride or fractal titanium nitride. Although the example shown in FIG. 3A includes two electrodes 313, in some embodiments, sensor device 310 can include 3, 4, 5, or more electrodes carried by the housing 314.

In the example shown in FIG. 3A, proximal end 322 includes a header assembly 332 that includes one or more of proximal electrode 313A, integrated antenna 326, anti-migration projections 334, or suture hole 336. Integrated antenna 326 is located on the same major surface (i.e., first major surface 318) as proximal electrode 313a and is also included as part of header assembly 332. Integrated antenna 326 allows sensor device 310 to transmit or receive data. In other examples, integrated antenna 326 may be formed on the opposite major surface as proximal electrode 313A, or may be incorporated within the housing 314 of sensor device 310. In the example shown in FIG. 3A, anti-migration projections 334 are located adjacent to integrated antenna 326 and protrude away from first major surface 318 to prevent longitudinal movement of the device. In the example shown in FIG. 3A anti-migration projections 334 includes a plurality (e.g., six or nine) small bumps or protrusions extending away from first major surface 318. As discussed above, in other examples, anti-migration projections 334 may be located on the opposite major surface as proximal electrode 313A or integrated antenna 326. In addition, in the example shown in FIG. 3A header assembly 332 includes suture hole 336, which provides another means of securing sensor device 310 to the patient to prevent movement following insert. In the example shown, suture hole 336 is located adjacent to proximal electrode 313A. In one example, header assembly 332 is a molded header assembly made from a polymeric or plastic material, which may be integrated or separable from the main portion of sensor device 310.

FIG. 3B shows a third electrode 392B at a midpoint between electrodes 390B and 391B. The dimension D of housing 374B of sensor device 360B can be increased to adjust the angle a to obtain a more orthogonal orientation for the triangular configuration of electrodes 390B-392B. In some examples, sensor device 360B may have the same shape and dimensions as sensor device 310, except that electrode 392B is added to the side surface or back surface of housing 374B to create a triangle-shaped electrode configuration. In addition, FIG. 3C shows sensor device 360 with an extended third dimension D. Third electrode 392C is positioned at a corner to create a triangular-shaped electrode configuration with electrodes 390C and 391C. Dimension D can be designed to achieve specific angles for the triangular configuration of electrodes 390C-392C. In some examples, sensor device 360B may be implanted at a location generally centered with respect to the thorax, the head, neck, or a target region. In some examples, sensor device 360B may be placed on an external surface of skin of a patient. In some examples, more than one sensor devices may be used to sense signals from the patient. For example, sensor device 360B may be implanted at cranial region for sensing EEG signals, and one or more sensor devices (e.g., on or more accelerometers) may be implanted at thorax region for sensing ECG signals and/or impedance. Such devices could communicate with each other and/or external device, and processing circuitry of one of the devices could determine stroke metric(s) based on the sensed signals and/or impedance.

FIG. 4 is a block diagram of an example configuration of a sensor device 400 configured to sense signals used to detect or predict a stroke of a patient. Sensor device 400 may be an example of any of sensor devices 210, 220, 230, 310, and 360B. In the illustrated example, sensor device 400 includes electrodes 418, antenna 405, processing circuitry 402, sensing circuitry 406, communication circuitry 404, storage device 410, switching circuitry 408, sensors 414 including motion sensor(s) 416, and power source 412.

Processing circuitry 402 may include fixed function circuitry and/or programmable processing circuitry. Processing circuitry 402 may include any one or more of a microprocessor, a controller, a DSP, an ASIC, an FPGA, or equivalent discrete or analog logic circuitry. In some examples, processing circuitry 402 may include multiple components, such as any combination of one or more microprocessors, one or more controllers, one or more DSPs, one or more ASICs, or one or more FPGAs, as well as other discrete or integrated logic circuitry. The functions attributed to processing circuitry 402 herein may be embodied as software, firmware, hardware or any combination thereof. Processing circuitry 402 may be an example of or component of processing circuitry 110 (FIGS. 1A and 1B).

Sensing circuitry 406 and communication circuitry 404 may be selectively coupled to electrodes 418A-418C via switching circuitry 408, as controlled by processing circuitry 402. Sensing circuitry 406 may monitor signals from electrodes 418A-418C in order to monitor electrical activity of the brain and heart (e.g., to produce an EEG and ECG) from which processing circuitry 402 (or processing circuitry of another device) may determine values over time of parameters used to generate the detection or prediction of stroke. Sensing circuitry 406 may also sense physiological characteristics such as subcutaneous tissue impedance, the impedance being indicative of at least some aspects of patient 102's tissue perfusion, ejection fraction, and/or other cardiovascular performance metrics. Tissue impedance may vary based on tissue perfusion, which may in turn vary based on ejection fraction and/or other cardiac performance metrics. In some examples, a sensor device may be configured to (e.g., have electrodes positioned and spaced to) measure other impedances that vary based on ejection fraction or other cardiac performance metrics, such as thoracic impedance. Degradation of ejection fraction, or other heart failure or other cardiac performance metrics, may be indicative of an increased risk of stroke.

With respect to tissue impedance indicative of cranial tissue perfusion, in some subjects, about twenty percent of all blood flow from the heart is channeled to the brain. This results in relatively stable tissue impedance measurements on or near the head when the brain is healthy. Relatively stable baseline tissue impedance measurements on or near the head may enable stroke detection based on deviations from these baselines resulting from changes in cranial tissue perfusion due to stroke. A significant change in the impedance values over a period of time associated with decreased stroke volume may be used by an algorithm (implemented by processing circuitry 402) as evidence of a suprathreshold likelihood of stroke.

Additionally, different changes the tissue impedance values may indicate different types of strokes. Processing circuitry 402 may classify stroke, e.g., as ischemic or hemorrhagic, based on determined tissue impedance values. For example, a sudden increase in impedance corresponding to reduced blood flow may indicate of an LVO (Large Vessel Occlusion) or ischemic stroke event (e.g., due to a blockage of cranial vasculature). Furthermore, a sudden decrease in impedance corresponding to blood pooling may indicate of an aneurism or hemorrhagic stroke event.

In some examples, an impedance signal collected by sensor device 400 may indicate respiratory patterns, e.g., a respiratory rate and/or a respiratory intensity, of patient 102. Sensing circuitry 406 also may monitor signals from sensors 414, which may include motion sensor(s) 416, and any additional sensors, such as light detectors, pressure sensors, or acoustic sensors, that may be positioned on or in sensor device 400. In some examples, respiratory patterns can be obtained via a blended sensor technique (ECG baseline shift plus impedance or 3-axis accelerometer vibration plus impedance). In some examples, sensing circuitry 406 may include one or more filters and amplifiers for filtering and amplifying signals received from one or more of electrodes 418A-418C and/or sensor(s) 414.

Communication circuitry 404 may include any suitable hardware, firmware, software or any combination thereof for communicating with another device, such as external device 108. Under the control of processing circuitry 402, communication circuitry 404 may receive downlink telemetry from, as well as send uplink telemetry to, external device 108 or another device with the aid of an internal or external antenna, e.g., antenna 405. In addition, processing circuitry 402 may communicate with a networked computing device via an external device (e.g., external device 108) and a computer network, such as the Medtronic CareLink® Network developed by Medtronic, plc, of Dublin, Ireland.

A clinician or other user may retrieve data from sensor device 400 using external device 108, or by using another local or networked computing device configured to communicate with processing circuitry 402 via communication circuitry 404. The clinician may also program parameters of sensor device 400 using external device 108 or another local or networked computing device.

In some examples, storage device 410 may be referred to as a memory and include computer-readable instructions that, when executed by processing circuitry 402, cause sensor device 400 and processing circuitry 402 to perform various functions attributed to sensor device 400 and processing circuitry 402 herein. Storage device 410 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 410 may also store data generated by sensing circuitry 406, such as signals, or data generated by processing circuitry 402, such as parameter values or indications of detections or predictions of stroke.

Power source 412 is configured to deliver operating power to the components of sensor device 400. Power source 412 may include a battery and a power generation circuit to produce the operating power. In some examples, the battery is rechargeable to allow extended operation. In some examples, recharging is accomplished through proximal inductive interaction between an external charger and an inductive charging coil within external device 108. Power source 412 may include any one or more of a plurality of different battery types, such as nickel cadmium batteries and lithium ion batteries. A non-rechargeable battery may be selected to last for several years, while a rechargeable battery may be inductively charged from an external device, e.g., on a daily or weekly basis.

As described herein, sensor device 400 may be configured to sense signals, e.g., via electrodes 418 and sensors 414, for detecting and predicting stroke. In some examples, processing circuitry 402 may be configured to calculate parameter values relating to one or more electrical signals received from the electrodes 418, and/or signals from sensors 414. In some examples, processing circuitry 402 may be configured to algorithmically determine whether the patient has a supra-threshold risk of stroke based on the parameter values.

In some examples, processing circuitry 402 may employ patient movement information as a part of the detection and prediction of stroke. For example, motion sensor 416 may include one or more accelerometers configured to detect patient movement. Processing circuitry 402 or sensing circuitry 406 may determine whether or not a patient has fallen based on the patient movement data collected via the accelerometer. Fall detection can be particularly valuable when assessing potential stroke patients, as a large percentage of patients admitted for ischemic or hemorrhagic stroke have been found to have had a significant fall within 15 days of the stroke event. Accordingly, in some embodiments, the processing circuitry 402 can be configured to initiate or modify a stroke detection or prediction algorithm upon fall (or near fall) detection using the accelerometer. In addition to fall detection, motion sensor 416 can be used to determine potential body trauma due to sudden acceleration and/or deceleration (e.g., a vehicular accident, sports collision, concussion, etc.). These events could cause a thrombolytic and/or plaque body to be dislodged , a precursor to stroke. Similar to stroke determination, these fall determinations or other movements can be employed by processing circuitry 402 when detecting or predicting a stroke.

FIG. 5 is a block diagram of an example configuration of an external device 500 configured to communicate with any sensor device (e.g., sensor device 106 or sensor device 400) described herein. External device 500 is an example of external device 108 of FIG. 1A. In the example of FIG. 5, external device 500 includes processing circuitry 502, communication circuitry 504, storage device 510, user interface 506, and power source 508.

Processing circuitry 502, in one example, may include one or more processors that are configured to implement functionality and/or process instructions for execution within external device 500. For example, processing circuitry 502 may be capable of processing instructions stored in storage device 510. Processing circuitry 502 may include, for example, microprocessors, DSPs, ASICs, FPGAs, or equivalent discrete or integrated logic circuitry, or a combination of any of the foregoing devices or circuitry. Accordingly, processing circuitry 502 may include any suitable structure, whether in hardware, software, firmware, or any combination thereof, to perform the functions ascribed herein to processing circuitry 502. Processing circuitry 502 may be an example of or component of processing circuitry 110 (FIGS. 1A and 1B).

Communication circuitry 504 may include any suitable hardware, firmware, software or any combination thereof for communicating with another device, such as IMD 400. Under the control of processing circuitry 502, communication circuitry 504 may receive downlink telemetry from, as well as send uplink telemetry to, sensor device 400, or another device.

Storage device 510 may be configured to store information within external device 500 during operation. Storage device 510 may include a computer-readable storage medium or computer-readable storage device. In some examples, storage device 510 includes one or more of a short-term memory or a long-term memory. Storage device 510 may include, for example, RAM, dynamic random access memories (DRAM), static random access memories (SRAM), magnetic discs, optical discs, flash memories, or forms of electrically programmable memories (EPROM) or EEPROM. In some examples, storage device 510 is used to store data indicative of instructions for execution by processing circuitry 502. Storage device 510 may be used by software or applications running on external device 500 to temporarily store information during program execution.

Data exchanged between external device 500 and sensor device 400 may include operational parameters. External device 500 may transmit data including computer readable instructions which, when implemented by sensor device 400, may control sensor device 400 to change one or more operational parameters and/or export collected data. For example, processing circuitry 502 may transmit an instruction to sensor device 400, which requests sensor device 400 to export collected data (e.g., data corresponding to one or more of the sensed signals, parameter values determined based on the signals, or indications that a stroke has been detected or predicted) to external device 500. In turn, external device 500 may receive the collected data from sensor device 400 and store the collected data in storage device 510. In some examples, external device 500 may provide an alert to the patient or another entity (e.g., a call center) based on a stroke detection or prediction provided by sensor device 400.

A user, such as a clinician or patient 102, may interact with external device 500 through user interface 506. User interface 506 includes a display (not shown), such as an LCD or LED display or other types of screen, with which processing circuitry 502 may present information related to IMD 400 (e.g., stroke metric). In addition, user interface 506 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 502 of external device 500 and provide input. In other examples, user interface 506 also includes audio circuitry for providing audible notifications, instructions or other sounds to patient 102, receiving voice commands from patient 102, or both. Storage device 510 may include instructions for operating user interface 506 and for managing power source 508.

Power source 508 is configured to deliver operating power to the components of external device 500. Power source 508 may include a battery and a power generation circuit to produce the operating power. In some examples, the battery is rechargeable to allow extended operation. Recharging may be accomplished by electrically coupling power source 508 to a cradle or plug that is connected to an alternating current (AC) outlet. In addition, recharging may be accomplished through proximal inductive interaction between an external charger and an inductive charging coil within external device 500. In other examples, traditional batteries (e.g., nickel cadmium or lithium ion batteries) may be used. In addition, external device 500 may be directly coupled to an alternating current outlet to operate.

FIG. 6 is a block diagram illustrating an example system that includes an access point 600, a network 602, external computing devices, such as a server 604, and one or more other computing devices 610A-610N, which may be coupled to sensor device 106, external device 108, and processing circuitry 110 via network 602, in accordance with one or more techniques described herein. In this example, sensor device 106 may use communication circuitry to communicate with external device 108 via a first wireless connection, and to communicate with an access point 600 via a second wireless connection. In the example of FIG. 6, access point 600, external device 108, server 604, and computing devices 610A-610N are interconnected and may communicate with each other through network 602.

Access point 600 may include a device that connects to network 602 via any of a variety of connections, such as telephone dial-up, digital subscriber line (DSL), or cable modem connections. In other examples, access point 600 may be coupled to network 602 through different forms of connections, including wired or wireless connections. In some examples, access point 600 may be a user device, such as a tablet or smartphone, that may be co-located with the patient. As discussed above, sensor device 106 may be configured to transmit data, such as signals, parameter values determined from signals, or stroke metric, to external device 108. In addition, access point 600 may interrogate sensor device 106, such as periodically or in response to a command from the patient or network 602, in order to retrieve such data from sensor device 106, or other operational or patient data from sensor device 106. Access point 600 may then communicate the retrieved data to server 604 via network 602.

In some cases, server 604 may be configured to provide a secure storage site for data that has been collected from sensor device 106, and/or external device 108. In some cases, server 604 may assemble data in web pages or other documents for viewing by trained professionals, such as clinicians, via computing devices 610A-610N. One or more aspects of the illustrated system of FIG. 6 may be implemented with general network technology and functionality, which may be similar to that provided by the Medtronic CareLink® Network developed by Medtronic plc, of Dublin, Ireland.

Server 604 may include processing circuitry 606. Processing circuitry 606 may include fixed function circuitry and/or programmable processing circuitry. Processing circuitry 606 may include any one or more of a microprocessor, a controller, a DSP, an ASIC, an FPGA, or equivalent discrete or analog logic circuitry. In some examples, processing circuitry 606 may include multiple components, such as any combination of one or more microprocessors, one or more controllers, one or more DSPs, one or more ASICs, or one or more FPGAs, as well as other discrete or integrated logic circuitry. The functions attributed to processing circuitry 606 herein may be embodied as software, firmware, hardware or any combination thereof. In some examples, processing circuitry 606 may perform one or more techniques described herein based on sensed signals and/or parameter values received from sensor device 106. For example, processing circuitry may perform one or more of the techniques described herein to detect and/or predict the risk of stroke of patient 102.

Server 604 may include memory 608. Memory 608 includes computer-readable instructions that, when executed by processing circuitry 606, cause server 604 and processing circuitry 606 to perform various functions attributed to server 604 and processing circuitry 606 herein. Memory 608 may include any volatile, non-volatile, magnetic, optical, or electrical media, such as RAM, ROM, NVRAM, EEPROM, flash memory, or any other digital media.

In some examples, one or more of computing devices 610A-610N (e.g., device 610A) may be a tablet or other smart device located with a clinician, by which the clinician may program, receive alerts from, and/or interrogate sensor device 106. For example, the clinician may access data corresponding to any one or combination of sensed physiological signals, parameters, or indications of detected or predicted strokes collected by sensor device 106. In some examples, the clinician may enter instructions for medical intervention for patient 102 into an app in device 610A, such as based on a stroke status determined by sensor device 106, external device 108, processing circuitry 110, or any combination thereof, or based on other patient data known to the clinician. Device 610A then may transmit the instructions for medical intervention to another of computing devices 610A-610N (e.g., device 610B or external device 108) located with patient 102 or a caregiver of patient 102. For example, such instructions for medical intervention may include an instruction to change a drug dosage, timing, or selection, to schedule a visit with the clinician, or to seek medical attention. In further examples, device 610B may generate an alert to patient 102 based on a stroke status of patient 102 determined by sensor device 106, which may enable patient 102 proactively to seek medical attention prior to receiving instructions for medical intervention. In this manner, patient 102 may be empowered to take action, as needed, to address his or her stroke status, which may help improve clinical outcomes for patient 102.

FIG. 7 is a flow diagram illustrating an example of operations for detecting and predicting strokes based on tissue impedance values detected via a plurality of electrodes of sensor devices, such as sensor devices 106, 210, 220, 310, 400, which are disposed at the neck, lower back of the head, or otherwise above the shoulders of a patient. The example technique of FIG. 7 is described as being performed by sensor device 400 and processing circuitry 110, but may be performed by any one or more sensor devices described herein, e.g., which may be configured as illustrated with respect to sensor device 400 in FIG. 4. As described herein, processing circuitry 110 may include processing circuitry of any one or more devices described herein, such as processing circuitry 402 of sensor device 400, processing circuitry 502 of external device 500, or processing circuitry 606 or server 604.

Sensor device 400 includes one or more sensors, such as electrodes 418 and sensors 414. Sensing circuitry 406 of sensor device 400 senses one or more electrical signals via electrodes 418. Sensing circuitry 406 may measure impedance values that represent ejection fraction, which measures the volume of blood left ventricle pumps out with each contraction of the heart of a patient. With each heartbeat, a certain amount of blood is pumped out of the heart of patient 102. Low blood volume may lead to low blood pressure, and organs and tissues may not receive enough blood to optimally and/or properly function, which may lead to stroke. Based on the impedance measurements, sensing circuitry 406 and/or processing circuitry 110 may determine one or a plurality of tissue impedance values that vary as a function of ejection fraction of the heart of patient 102 (702).

In some examples, the electrical signals may further include a brain electrical signal (e.g., an EEG signal) and a cardiac or heart electrical signal (e.g., an ECG signal). The sensed signals may also include a motion signal sensed by motion sensor 416, e.g., one or more accelerometers. The sensed signals may also include respiration signals, skin impedance signals, and/or perfusion signals (e.g., sensed via impedance using electrodes 418), blood pressure signals (e.g., sensed via photoplethysmography using optical sensors), heart sound signals (e.g., sensed using motion sensor 416 or an acoustic sensor), evoked potentials (e.g., response from electrical stimulus) or ballistocardiogram signals (e.g., sensed using the ECG and motion sensor signals).

The signals, tissue impedance values, or parameters derived therefrom, may be useful for detecting and predicting strokes of a patient. For example, an impedance, a brain electrical signal, and a cardiac electrical signal may be useful for detecting or predicting stroke. Additional parameters and signals may improve the sensitivity and specificity of the detection and prediction of stroke by processing circuitry 110.

The example technique of FIG. 7 may include pre-processing and parameter value extraction, which may be performed by sensing circuitry 406 and/or processing circuitry 110. Pre-processing may include any of a variety of analog and/or digital filtering or other signal processing techniques to allow ready extraction of values of the desired features or parameters from a signal. Processing circuitry 110 then determines, based on the parameter values and/or signals, a stroke metric indicative of a stroke status of patient 102.

In some examples, processing circuitry 110 may determine the stroke metric indicative of a stroke status of patient 102 based on the impedance measurements. According to the example of FIG. 7, processing circuitry 110 may determine one or a plurality of tissue impedance values that vary as a function of ejection fraction of the heart of patient 102. A significant change in the impedance values over a period of time associated with decreased stroke volume may be used by an algorithm as evidence of a suprathreshold likelihood of stroke. Additionally, a sudden increase in impedance corresponding to reduced blood flow may indicate of an LVO (Large Vessel Occlusion) or ischemic stroke event. Furthermore, a sudden decrease in impedance corresponding to blood pooling may indicate of an aneurism or hemorrhagic stroke event. Processing circuitry 110 may then determine the stroke metric based on the one or plurality of tissue impedance values, and in some cases other patient parameters (e.g., change in EEG, ECG, and/or accelerometry values) (704). Processing circuitry 110 may further store the stroke metric in a memory, such as storage device 410.

In some examples, processing circuitry 110 may determine the stroke metric indicative of a stroke status of patient 102 based on the brain electrical signal (e.g., EEG signals). Processing circuitry 110 may determine brain activity data based on an EEG signal. For example, processing circuitry 110 may determine a power of the brain electrical signal within certain selected frequency bands and determine the stroke metric based on both of the power of the brain electrical signal and the plurality of tissue impedance values.

In some examples, processing circuitry 110 may determine the stroke metric indicative of a stroke status of patient 102 based on the cardiac electrical signal (e.g., ECG signals). Processing circuitry 110 may determine heart activity data based on an EEG signal. For example, processing circuitry 110 may further identify beats within the cardiac electrical signal and determine the stroke metric based on both beats within the cardiac electrical signal and the plurality of tissue impedance values.

In some examples, processing circuitry 110 may determine the stroke metric indicative of a stroke status of patient 102 based on motion data detected via an accelerometer. For example, processing circuitry 110 may use motion data as a weighted factor to determine the stroke metric based on both the motion data and the plurality of tissue impedance values (e.g., the patient falls and show no motion after a stroke event may be given greater weight than if the patient falls and posture/activity shows upright and walking around after a stroke event).

Techniques for using brain electrical signal, cardiac electrical signal, or motion data for determining patient conditions, such as stroke, are described in U.S. Provisional Patent Application No. 63/071,908, filed on Aug. 28, 2020, and titled “DETECTION OF PATIENT CONDITIONS USING SIGNALS SENSED ON OR NEAR THE HEAD” (ATTY DOCKET NO. A0005021US01/1213-130USP1), the entire content of which is incorporated herein by reference.

Processing circuitry 110 may employ various techniques to determine the stroke metric. For example, processing circuitry 110 may generate the stroke metric using one or more different algorithms, such as using machine learning algorithms.

In some examples, processing circuitry 110 may compare the stroke metric with a respective stroke threshold that indicates a stroke is occurring or has occurred (706). In this manner, processing circuitry 110 may provide an alert when the stroke metric is greater than or equal to the stroke threshold (710). For example, processing circuitry 110 may send an alert to an external device to inform patient 102 or a clinician that the patient may need assistance or therapeutic intervention. Processing circuitry 110 continues to sense electrical signals from patient 102 when the stroke metric is less than the stroke threshold (708).

When processing circuitry 110 transmits the stroke metric to an external device, the external device may be associated with emergency services in some examples. In some examples, the external device may include global position system (GPS) capability or other location detection technology (e.g., WiFi triangulation) such that the external device can identify, store, and/or communicate the geographic location at which the stroke metric occurred. The external device may then transmit the location information and/or stroke metric to another device or system via cell phone tower, satellite, or other technology. The other system may be an emergency service such as 911 or other medical services. If the technique of FIG. 7 is performed in an ambulance, for example, a device carried by ambulance or technician may receive the metric and output information or instructions to an emergency medical technician (EMT) or other personnel in the rear of the ambulance and/or to the ambulance driver. In some examples, the display to the ambulance driver can include navigational information such as a map and instructions to take patient 102 to a particular hospital or facility with a stroke center or stroke expertise.

FIG. 8 is a flow diagram illustrating another example of operations for detecting and predicting strokes based on one or a plurality of tissue impedance values detected via a plurality of electrodes of sensor devices, such as sensor devices 106, 210, 220, 310, 400, which are disposed at the neck, lower back of the head, or otherwise above the shoulders of a patient. The example technique of FIG. 8 is described as being performed by sensor device 400 and processing circuitry 110, but may be performed by any sensor device described herein, e.g., which may be configured as illustrated with respect to sensor device 400 in FIG. 4. As described herein, processing circuitry 110 may include processing circuitry of any one or more devices described herein, such as processing circuitry 402 of sensor device 400, processing circuitry 502 of external device 500, or processing circuitry 606 or server 604.

Sensing circuitry 406 of sensor device 400 senses one or more electrical signals via electrodes 418. The electrical signals may include an electrical signal that represents ejection fraction, which measures the volume of blood left ventricle pumps out with each contraction of the heart of a patient. According to the example of FIG. 8, processing circuitry 110 may determine one or a plurality of tissue impedance values that vary as a function of ejection fraction of the heart of patient 102 based on the ejection fraction electrical signal sensed during a first time period. A significant change in the impedance values over a period of time associated with decreased stroke volume may be used by an algorithm as evidence of a suprathreshold likelihood of stroke. Additionally, a sudden increase in impedance corresponding to reduced blood flow may indicate of an LVO (Large Vessel Occlusion) or ischemic stroke event. Furthermore, a sudden decrease in impedance corresponding to blood pooling may indicate of an aneurism or hemorrhagic stroke event. Processing circuitry 110 may then determine the stroke metric based on the one or plurality of tissue impedance values, and in some cases other patient parameters (e.g., change in EEG, ECG, and/or accelerometry values) (704). Processing circuitry 110 may then determine a first stroke metric based on the one or plurality of tissue impedance values during the first time period (802).

According to the example of FIG. 8, processing circuitry 110 may also determine one or a plurality of tissue impedance values that vary as a function of ejection fraction of the heart of patient 102 based on the ejection fraction electrical signal sensed during a second time period. Processing circuitry 110 may then determine a second stroke metric based on the one or plurality of tissue impedance values during the second time period (804).

Processing circuitry 110 may then compare the second stroke metric for the second time period to the first stroke metric for the first time period (806). If the value for the second stroke metric remained the same (i.e., did not increase or decrease) (808) relative to the first stroke metric, processing circuitry 110 may determine a stroke metric for the next time period. However, if the value for the second stroke metric has varied (e.g., increased or decreased) beyond a threshold value (810), processing circuitry 110 may determine a sudden change in the stroke metric has occurred and send an alert to an external device to inform patient 102 or a clinician that the patient may need assistance or therapeutic intervention.

FIG. 9 is a flow diagram illustrating an example of operations for detecting and predicting strokes based on clinical characteristics and tissue impedance values detected via a plurality of electrodes of sensor devices. The example technique of FIG. 9 is described as being performed by sensor device 400 and processing circuitry 110, but may be performed by any sensor device described herein, e.g., which may be configured as illustrated with respect to sensor device 400 in FIG. 4. As described herein, processing circuitry 110 may include processing circuitry of any one or more devices described herein, such as processing circuitry 402 of sensor device 400, processing circuitry 502 of external device 500, or processing circuitry 606 or server 604.

According to the example of FIG. 9, processing circuitry 110 may obtain clinical data of patient 102 (902). The clinical data may represent clinical symptoms that are presented during a stroke. For example, posture has an important impact on cardiovascular stress and the autonomic nervous system, which may precipitate certain conditions, such as stroke. Sensor device 400 and/or an external device (e.g., external device 108) may capture posture, motion, respiration and other sensor signals, which represent clinical symptoms that are present during stroke events.

In some examples, processing circuitry 110 may receive clinical data of patient 102 via external device 108. For example, external device 108 may capture clinical data of patient 102 (e.g., the patient's activity or condition in response to prompts, questions or other stimuli) using a camera (e.g., to detect facial drooping), a microphone (e.g., to detect slurred speech), or to detect any other indicia of stroke. Additionally or alternatively, processing circuitry 110 may receive clinical data of patient 102 collected via sensor device 400. For example, external device 108 may instruct the user to lift an arm, make a facial expression, etc., and sensor device 400 may record physiological data while the user performs the requested actions.

According to the example of FIG. 9, processing circuitry 110 may extract one or more clinical characteristics from the clinical data (904). The one or more extracted clinical characteristics may include speech characteristics (e.g., syllables, intonation, etc.), facial expression characteristics (e.g., asymmetric response or expression, such as eyelid droop, lip droop, facial numbness, etc.), and other clinical characteristics (e.g., the National Institutes of Health Stroke Scale (NIHSS), the Cincinnati Prehospital Stroke Scale (CPSS), the Los Angeles Prehospital Stroke Screen (LAPSS), etc.) to determine whether a stroke event has occurred.

According to the example of FIG. 9, processing circuitry 110 may determine a stroke metric indicative of a stroke status of patient 102 based on the extracted clinical characteristics and one or a plurality of tissue impedance values representative of ejection fraction of the heart of patient 102 (906). For example, extracted clinical characteristics can be compared against pre-stroke inputs (e.g., a stored baseline facial image or voice-print with baseline speech recording) to generate a weighted score. Processing circuitry 110 may further apply the weighted score to a stroke score determined based on the plurality of tissue impedance values to generate a stroke metric. Processing circuitry 110 may then compare the stroke metric with a respective stroke threshold that indicates a stroke is occurring or has occurred.

In some examples, a normative profile may be used to generate the stroke threshold. FIG. 10 is a flow diagram illustrating an example of operations for generating a stroke threshold based on a normative profile, in accordance with one or more aspects of this disclosure.

According to the example of FIG. 10, processing circuitry 110 may obtain patient profile information of patient 102 (1002). Patient profile information of patient 102 may include age, gender, health condition, fitness level, stroke history, stroke diagnosis, types or origins of stroke (e.g., ischemic or hemorrhagic, or which hemisphere, for stroke), treatment type, and treatment duration of patient 102.

Processing circuitry 110 may select a normative profile based on the patient profile information of patient 102 (1004). This disclosure refers to a normative profile to a caustic profile, which is known to be representative or which is associated with a specific type of stroke. In some examples, such a normative profile can be compiled from normalizing or averaging patient profile information of a number of patients with a common type of stroke. In some examples, processing circuitry 110 may select the normative profile from a plurality of normative profiles based on the patient profile information of patient 102 matches at least a portion of the selected normative profile. Processing circuitry 110 may then determine a stroke threshold that indicates a stroke is occurring or has occurred based on the selected normative profile (1006).

FIG. 11 is a conceptual diagram of another example system 1100 in conjunction with a patient 1102, in accordance with one or more techniques of this disclosure. Medical system 1100 may be substantially similar to medical systems 100A and 100B of FIGS. 1A and 1B, except as noted herein. For example, medical system 1100 may include a sensor device 1106A configured to be implanted or otherwise positioned at a target location 1104, an external device 1108, and processing circuitry 1110, which may be similar to the like numbered elements of FIGS. 1A-6. Sensor device 1106A may correspond to any of sensor devices 106, 210, 220, 230, 240, 250, 310, 360, and 400 described herein.

System 1100 additionally includes a sensor device 1106B, which may be implanted or otherwise positioned at a different location of patient than target location 1104. For example, sensor device 1106B may be implanted subcutaneously in a pectoral region of patient 1102. Sensor devices 1106A and 1106B may include respective electrodes and, in some examples, respective other sensors to sense respective physiological signals. For example, sensor device 1106A may be configured to sense EEG, motion, and impedance signals, while sensor device 1106B is configured to sense ECG and motion signals. Processing circuitry 1110, e.g., of external device 1108, may derive data from the signals, and apply an algorithm to the data to detect or predict stroke as described herein. As described above, in some examples external device 1108 may be a smartphone or smartwatch of patient 1102.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or an external programmer.

What is claimed is:

1. A system comprising:
a memory;
a plurality of electrodes;
sensing circuitry configured to:
determine one or more tissue impedance values via the electrodes, wherein the tissue impedance values vary as a function of ejection fraction of a heart of a patient; and
processing circuitry configured to:
determine, at least based on the one or more tissue impedance values, a stroke metric indicative of a stroke status of the patient; and
store the stroke metric in the memory.

2. The system of claim 1, wherein the processing circuitry is configured to:
compare the stroke metric to a stroke threshold; and
output an alert in response to the stroke metric satisfying the stroke threshold.

3. The system of claim 1, wherein the processing circuitry is configured to:
determine, at least based on a first set of tissue impedance values of the one or more tissue impedance values during a first period, a first stroke metric;
determine, at least based on a second set of tissue impedance values of the one or more tissue impedance values during a second period, a second stroke metric;
compare the second stroke metric to the first stroke metric to determine whether a sudden change in the stroke metric has occurred; and
output an alert in response to a determination the sudden change in the stroke metric has occurred.

4. The system of claim 1, wherein the sensing circuitry is configured to determine the one or more tissue impedance values by at least sensing an electroencephalogram (EEG) signal via the plurality of electrodes, and wherein the processing circuitry is configured to:
generate brain activity data based on the EEG signal; and
determine the stroke metric based on the brain activity data.

5. The system of claim 1, wherein the sensing circuitry is configured to determine the one or more tissue impedance values by at least sensing an electrocardiogram (ECG) signal via the plurality of electrodes, and wherein the processing circuitry is configured to:
generate heart activity data based on the ECG signal; and
determine the stroke metric based on the heart activity data.

6. The system of claim 1, further comprising an accelerometer configured to generate motion data representative of motion of the patient, and wherein the processing circuitry is configured to:
determine the stroke metric based on the motion data.

7. The system of claim 6, wherein the processing circuity is further configured to:
determine, based on the motion data, that the patient has fallen; and
determine the stroke metric based on the determination that the patient has fallen.

8. The system of claim 1, wherein the processing circuity is configured to:
obtain clinical data of the patient;
extract clinical characteristics from the clinical data, wherein the clinical characteristics comprises at least one of speech characteristics or facial expression characteristics; and
determine the stroke metric based on the clinical characteristics.

9. The system of claim 8, further comprising an implantable medical device comprising the plurality of electrodes and the sensing circuitry, wherein the processing circuity is configured to receive at least some of the clinical data from an external device.

10. The system of claim 2, wherein the processing circuity is further configured to:
select a normative profile from a plurality of normative profiles, wherein at least a portion of the selected normative profile matches patient profile information of the patient; and
generate the stroke threshold based on the selected normative profile.

11. The system of claim 1, further comprises a housing carrying the plurality of electrodes and containing both of the sensing circuitry and the processing circuitry.

12. The system of claim 11, wherein the housing is configured to be disposed at or adjacent region of a thorax, a rear portion of a neck, or skull base of the patient.

13. The system of claim 11, wherein the housing is configured to be implanted within the patient.

14. The system of claim 11, wherein the housing is configured to be implanted subcutaneously.

15. The system of claim 1, further comprising:
a housing containing both of the sensing circuitry and at least some of the processing circuitry; and
at least one sensing extension coupled to the housing and carrying at least one electrode of the plurality of electrodes.

16. The system of claim 1, wherein the plurality of electrodes comprises a first plurality of electrodes and the sensing circuitry comprises first sensing circuitry, the system further comprising:
a first implantable medical device comprising the first plurality of electrodes and the first sensing circuitry;
a second implantable medical device comprising a second plurality of electrodes and second sensing circuitry configured to sense an electrocardiogram of the patient via the second plurality of electrodes; and
an external device, wherein the processing circuitry comprises processing circuitry of the external device configured to determine the stroke metric based on the one or more tissue impedance values and the electrocardiogram signal.

17. A method comprising:
determining, via a plurality of electrodes, one or more tissue impedance values, wherein the tissue impedance values vary as a function of ejection fraction of a heart of a patient;
determining, via processing circuitry and at least based on the one or more tissue impedance values, a stroke metric indicative of a stroke status of the patient; and
storing the stroke metric in a memory.

18. The method of claim 17, further comprising:
comparing, by the processing circuitry, the stroke metric to a stroke threshold; and
outputting an alert in response to the stroke metric satisfying the stroke threshold.

19. The method of claim 17, further comprising:
determining, by the processing circuitry and at least based on a first set of tissue impedance values of the one or more tissue impedance values during a first period, a first stroke metric;
determining, by the processing circuitry and at least based on a second set of tissue impedance values of the one or more tissue impedance values during a second period, a second stroke metric;
comparing, by the processing circuitry, the second stroke metric to the first stroke metric to determine whether a sudden change in the stroke metric has occurred; and
outputting, by the processing circuitry, an alert in response to a determination the sudden change in the stroke metric has occurred.

20. The method claim 17, further comprising:
sensing an electroencephalogram (EEG) signal via the plurality of electrodes;
generating, by the processing circuitry, brain activity data based on the EEG signal; and
determining, by the processing circuitry, the stroke metric based on the brain activity data.

21. The method claim 17, further comprising:
sensing an electrocardiogram (ECG) signal via the plurality of electrodes;
generating, by the processing circuitry, heart activity data based on the ECG signal; and
determining, by the processing circuitry, the stroke metric based on the heart activity data.

22. The system of claim 17, further comprising:
generating motion data representative of motion of the patient, and
determining, by the processing circuitry, the stroke metric based on the motion data.

23. The method of claim 22, further comprising:
determining, by the processing circuitry and based on the motion data, that the patient has fallen; and
determining, by the processing circuitry, the stroke metric based on the determination that the patient has fallen.

24. The method of claim 17, further comprising:
obtaining, by the processing circuitry, clinical data of the patient;
extracting, by the processing circuitry, clinical characteristics from the clinical data, wherein the clinical characteristics comprises at least one of speech characteristics or facial expression characteristics; and
determining, by the processing circuitry, the stroke metric based on the clinical characteristics.

25. The method of claim 17, wherein the an implantable medical device comprises the plurality of electrodes, the method further comprising:
receiving at least some of the clinical data from an external device.

26. The method of claim 18, the method further comprising:
selecting a normative profile from a plurality of normative profiles, wherein at least a portion of the selected normative profile matches patient profile information of the patient; and
generating the stroke threshold based on the selected normative profile.
